(19) United States
(12) Patent Application Publication
(10) Pub. No.: US 2016/0377635 A1
(43) Pub. Date: Dec. 29, 2016

(54) Measurement of Oxytocin and Vasopressin

(71) Applicant: Martin-Protean LLC, Princeton, NJ (US)

(72) Inventor: Warham Lance Martin, JR., Chicago, IL (US)

(21) Appl. No.: 14/901,565

(22) PCT Filed: Jun. 26, 2014
(86) PCT No.: PCT/US2014/044467

§ 371 Date: Dec. 28, 2015

Related U.S. Application Data
(60) Provisional application No. 61/839,627, filed on Jun. 26, 2013.
(60) Provisional application No. 61/846,947, filed on Jul. 16, 2013.

(51) Int. Cl. G01N 33/74; B01D 15/12; G01N 30/08; G01N 1/40

(52) U.S. Cl. CPC G01N 33/74; G01N 1/405; B01D 15/12; G01N 30/08; G01N 2333/575; G01N 2030/067

(57) ABSTRACT

The disclosure provides methods for processing a biological samples and determining the presence or an amount of a polypeptide in a biological sample, such as when the polypeptide is oxytocin or vasopressin.

### BACKGROUND OF THE INVENTION

Field of the Invention

Uncertainty surrounding the measurement and function of plasma oxytocin can be resolved by quantifying all plasma oxytocin forms using liquid chromatography and tandem mass spectrometry (LCMSn) under conditions that anticipate and manage oxytocin disulfide exchange. The current market for oxytocin quantification is limited sharply by the absence of a satisfactory measurement to academic research labs.

Description of Related Art

Oxytocin is the neuropeptide hormone responsible for inducing parturition and lactation. Exogenous oxytocin administered to human subjects increases trust between individuals and trust and self-sacrifice for in group members as well as defensive aggression towards those outside the group. Exogenous oxytocin also increases parasympathetic control of heart rate. Endogenous oxytocin levels are linked to successful wound healing, but measurements of endogenous oxytocin performed with commercially available immunoassays are fraught with uncertainty. Human plasma oxytocin concentrations obtained with immunoassays (1-500 pg/mL), especially those generated from the supernatent of an acetone precipitation of plasma (1-10 pg/mL) are curiously low relative to doses required for effect in administration to humans and relative to levels required in biological activity assays. Pitocin (synthetic oxytocin) intravenous dosing for labor starts at 400 pg/mL of total blood volume and is escalated to as much as 24 ng/mL for total blood volume to induce normal labor. The intranasal dose employed to induce trust would generate a 9.6 ng/mL concentration in the total blood volume. Similarly, the linear response range for oxytocin generating a contraction response from excised and bathed section of mammary gland tissue is from 1-20 ng/mL. This is 40-200 fold higher than is obtained with immuno assays.

Existing measurements of endogenous oxytocin quantify oxytocin by quantifying enzyme amplified or radioactively-labeled immunoreactivity to polyclonal anti-oxytocin antisera in subject samples, usually plasma. There is a single numerical readout that describes the intensity of the immunoreactivity in the sample. The different immunoassay measurements of oxytocin do not correlate with each other, they differ by orders of magnitude depending on plasma fractionation protocol and most damning both immunoassays reactive positively to multiple distinct plasma fractions obtained from reverse phase high-pressure liquid chromatography (HPLC) separation of plasma. Both immunoassays are bundling the responses to the distinct molecules that must occupy these distinct fractions and calling the whole response oxytocin. Fractions that comigrate with injected pure oxytocin are responsible for <20% of the total signal (Szeto, et al. (2011). Evaluation of enzyme immunoassay and radioimmunoassay methods for the measurement of plasma oxytocin. Psychosomatic medicine, 73(5), 393-400).

The difficulties measuring oxytocin with commercially available immunoassays are amplified by the use of polyclonal antibodies. Polyclonal antibodies have by definition multiple distinct molecules with anti-oxytocin immunoreactivity. Different antibodies in polyclonal antisera will bind oxytocin with different affinities. If there are any variations in the form of oxytocin it will not be possible to detect those differences with polyclonal antibodies, the antibodies generate too many different responses (none that are defined) and the responses are bundled into one readout. Further, any particular preparation of polyclonal antibodies is temporary, the antisera runs out and a new, different antisera must be obtained. So results obtained with polyclonal antisera at one time are compared with results obtained with a different prep with a significant limit on the possible conclusions.

Some fundamental problems associated with measuring oxytocin with polyclonal antibodies would be reduced considerably by using a monoclonal anti-oxytocin antibody. A monoclonal antibody is a single molecule and it may be obtained indefinitely from an immortalized cell-line. To date monoclonal antibodies exist for oxytocin (eg. 4G11) but their use is not wide spread and there are reports of confounding issues. Under standard immunoprecipitation conditions the leakage of oxytocin is significant and more than 80% of loaded oxytocin is not recovered (Mclaughlin, et al. (2008). Quantitative analysis of oxytocin and vasopressin by LC-MS/MS. 56th ASMS Conference on Mass Spectrometry (p. 8). Denver: Stanford University Mass Spectrometry Facility). There are no commercially available monoclonal antibodies for oxytocin-gkr (the long extended form) and cross-reactivity for oxytocin-gkr is anticipated but undefined. If there were ideal monoclonal antibodies against oxytocin and oxytocin-gkr and there was no reason to anticipate disulfide exchange, then they would be useful for generating oxytocin enriched fractions from biological samples. But antibody binding by itself would fail to produce the molecular detail, let alone that detail in every experiment, necessary to resolving oxytocin quantification ambiguity. Further, because having to manage oxytocin disulfide exchange with reducing agents, denaturants, detergents and modification reagents, all of which are incompatible with antibody-ligand binding, low utility from monoclonal antibodies for quantifying oxytocin is expected even if excellent ones are available.

The absence of molecular detail in the immunoassay quantifications for oxytocin is inadequate to characterize the multiple immunoreactive fractions obtained in published experiments (Szeto et al., 2011). It may be that in addition to the cyclic disulfide-containing carbamidated nonapeptide hormone oxytocin with the amino acid sequence CYIQNCPLG (SEQ ID: 2) that is the final product of oxytocin post-translational modification there also circulates in the blood the disulfide containing dodecapeptide CYIQNCPLGGKR (SEQ ID: 3) that is product of the first step of post-translational modification releasing oxytocin from the rest of the neurophysin protein it is expressed with. This might explain the multiple oxytocin immunoreactive fractions in HPLC fractionated plasma. However, it would not explain why immunoassay protocols that measure whole plasma produce oxytocin plasma concentrations that are usually 100-fold higher than protocols that measure the supernatent of an acetone precipitation of plasma (Szeto et al., 2011). Similarly, the presence of oxytocin-gkr would not explain why oxytocin quantifications of acetone precipitated plasma after oxytocin administration follow fast first order exponential decay kinetics while immunoassay measurements of whole plasma decrease slightly to a much higher level and oscillate randomly (Robinson et al. (2013). Validation of an enzyme-linked immunoassay (ELISA) for plasma oxytocin in a novel mammal species reveals potential errors induced by sampling procedure. J Neurosci Methods. 226; 73-79). It would also not explain the published observation that the majority of immunoreactive activity in plasma fractionated by size-exclusion chromatography is a found in a fraction containing high molecular weight proteins (30,000-50,000 Daltons, oxytocin and oxytocin-gkr are 1,000 Da) (Szeto et al., 2011).

### SUMMARY OF THE INVENTION

Whether multiple oxytocin immunoreactive fractions result from distinct oxytocin post-translational processing peptides or peptides generated by disulfide exchange or both, reliable oxytocin quantification requires specific molecular detail validating oxytocin measurements. Liquid chromatography tandem mass spectrometry (LCMSn) is singular among existing techniques in its ability to provide that detail from the small amounts present in partially purified assay samples. LCMSn resolves, identifies and quantifies target compounds in each experiment. As a mixture of compounds is injected onto the column in the liquid chromatography experiment, the compounds are separated as they progress down the column based on how the compounds interact with the column material. In ultraperformance liquid chromatography (UPLC), which can be used in the disclosure of the invention, it is possible to resolve oxytocin from the highly homologous peptide vasopressin with three minutes of separation from baseline to baseline, on conventionally steep gradients.

The challenge for quantifying oxytocin with LCMSn comes in generating an oxytocin enriched fraction from the sample for injection into the instrument. An LCMSn instrument of reasonable quality can detect 0.1 femtomole of oxytocin, or the oxytocin from 100 μL of plasma at 1 pg/mL. Virtually no HPLC assembly let alone a nanocapillary UPLC can handle the injection of 100 μL of plasma without instant and permanent column failure. If the column were somehow not to fail, it would not be possible with any available columns to resolve the oxytocin from all the other components of the plasma, even if it were present in reasonable abundance, plasma is too heterogeneous. Finally, the most abundant peptides in plasma (glutathione, albumin) are present at millimolar concentration and to achieve measurements of oxytocin in the picomolar range a billion-fold excess of extraneous material needs to be removed to uncover the target of interest.

In the methods of the disclosure, the surprisingly high levels of oxytocin and vasopressin are isolated from plasma after perturbing the disulfide equilibrium when the physical state of the plasma is “ideal.” This is a reproducible but challenging phenomenon to achieve, especially because plasma samples often start off with different physical states.

In one aspect, the disclosure provides methods for processing a biological sample, comprising:

contacting the sample with one or more of reducing agents, denaturants, detergents, modification reagents, and/or agents capable of catalyzing oxidation;
precipitating the high-molecular weight substituents of the sample with organic solvent; evaporation of the organic solvent; and
subjecting the aqueous remainder of the supernatent to solid phase extraction.

In another aspect, the disclosure provides methods for determining the presence or an amount of a polypeptide in a biological sample, the method comprising:

a. adding to the sample a decoy polypeptide
b. contacting the sample with a reducing agent and an agent capable of catalyzing oxidation;
c. isolating the polypeptide and decoy from the sample, and
d. detecting the polypeptide in the eluent.

In another aspect, the disclosure provides methods for determining the presence or an amount of a polypeptide in a biological sample, the method comprising:

a. contacting the sample with a reducing agent and an agent capable of catalyzing oxidation to obtain a reduced polypeptide;
b. contacting the reduced polypeptide with an alkylating agent to obtain an alkylated polypeptide;
c. isolating the alkylated polypeptide from the sample, and
d. detecting the polypeptide in the eluent.

### BRIEF DESCRIPTION OF DRAWINGS

FIG. 1 shows the recovery of oxytocin and vasopressin.

FIG. 2A-E images show the results of MS of the analog peptides, plasma with the addition of the analogs, plasma in which has subject to the foregoing process, the decoy peptide, and buffer blank and another run showing the how the proteolysis generated the correct decoys. (A) Analog preparation, (B) plasma plus process, (C) plasma without process, (D) plasma with process and decoy, and (E) decoy only.

FIG. 3A-C show measuring oxytocin and vasopressin in plasma using liquid chromatography tandem mass spectrometry (LC-MS/MS). (A) shows peak at 543 Vasopressin (CYFQNCPRG+2H+)/2 (SEQ ID. 1); at 1007 Oxytocin (CYIQNCPLG+H+)/1 (SEQ ID: 2); UPLC tandem MS instrumentation showing column resolution of oxytocin retention time ˜13 minutes, m/z ˜1007.5) from vasopressin (retention time ˜9.5 minutes, m/z ˜543.0, m=M2H+=1085.5, z=2). (B) shows peak at 543 (CYFQNCPRG+2H+)/2 (SEQ ID: 1); at 1007 (CYIQNCPLG+H+)/1 (SEQ ID: 2); UPLC tandem MS instrumentation showing mass resolution of oxytocin (m/z ˜1007.5, oxytocin exact mass=1006.3, proton mass=1, m=1007.5, z=1) from vasopressin (m/z ˜543.0, vasopressin exact mass 1083.5, two proton mass=2, m=1085.5, z=2). (C) shows peak at 328 (PRG+H+)/1; at 534 (CYFQNCPRG-OH+2H+)/2 (SEQ ID:1); at 757 (CYFQNC+H+)/1 (SEQ ID: 4); at 723 (CYIQNC+H+)/1 (SEQ ID: 5); at 990 (CYIQNCPLG-OH+H+)/1 (SEQ ID: 2); UPLC tandem MS instrumentation showing tandem msms fragmentation footprints of vasopressin (top) and oxytocin (bottom).

FIG. 4A shows a disulfide exchange, which is a chemical reaction that is used in biological systems for everything from folding proteins to modulating enzyme activity to transferring information. The basic for the reaction is R₁—SH+R₂—S—S—R₂<->R₁—S—S—R₂+R₂—SH.

FIG. 4B-C show disulfide exchange of oxytocin and vasopressin with glutathione at its plasma concentration in the presence of air (as in blood) and undergo additional oxidation resulting in the accumulation of diglutathione adducts. The identity of the adducts by msms fragmentation is confirmed. The adducts are equilibrium are seen in measurements by infusion ionization of the whole reactions without column purification (not shown).

FIG. 5 shows that incubating oxytocin and vasopressin with the free thiol containing albumin at pH 7.4, at room temperature (or 37° C.) with or without glutathione and cysteine leads to quantitative adsorption to albumin. Top: o oxytocin or vasopressin in supernatant above albumin after acetone precipitation of co-incubation (here with glutathione and cysteine as well). Bottom: No oxytocin or vasopressin in supernatant above albumin after acetone precipitation of coincubation (here without glutathione and cysteine).

FIG. 6 shows exogenous oxytocin and vasopressin eluted from acetone extraction pellets (middle) and isolated from plasma (top) at nM concentrations if plasma redox state is perturbed.

FIG. 7 shows endogenous oxytocin and vasopressin maybe isolated from plasma at nM concentrations if plasma redox state is perturbed.

FIG. 8 shows decoy peptides that are capable of participating equally with target compounds in both disulfide exchange and protein binding.

FIG. 9 shows treatment of endogenous oxytocin (nM) isolated by redox perturbation of whole plasma, with and without the decoy molecule.

FIG. 10 shows the LCMS peaks and LCMSMS fragmentation for the covalently modified forms of oxytocin, vasopressin and carboxy-terminal glycine deleted hormone analogs formed using the inventive method.

FIG. 11 shows the sensitivity of the hormone recovery to the amount of beta mercaptoethanol added prior to performing the extractions (2 extractions/solvent).

FIG. 12 shows the sensitivity of the hormone recovery to the amount of beta mercaptoethanol added prior to performing the extractions (3 extractions/solvent).

FIG. 13 shows the 25 pM current limit of quantification in plasma using this method as well as the near negligible ions suppression of hormone isolated from plasma relative to water.

### DETAILED DESCRIPTION OF THE INVENTION

The best alternative to quantifying hormone-competed immunoreactivity is to quantify the compound analytically. Analytical quantification of a molecule involves obtaining absolute evidence of compound identity as well as signal intensity that reports compound concentration. Liquid chromatography tandem mass spectrometry produces a retention time, a parent ion mass and a ion fragmentation fingerprint for absolute compound identification and the strength of the parent ion mass signal reports the compound concentration.

The analytical quantification of low abundance compounds from complex biological samples exceeds current capability (Wisniewski, et al., J Med Chem 2014). At picomolar concentrations, target abundance requires processing near milliliter quantities of sample to reach the minimal levels for detection on modern instrumentation. Capillary columns necessary for the most sensitive measurements cannot tolerate even nanoliter injections of unprocessed biological samples. Further, for successful quantification a picomolar peptide must be enriched relative to chemically similar but millimolar peptides and proteins in the sample a billion-fold.

Many outstanding solid phase extraction techniques exist for generating sufficiently enriched compounds for analysis from complex samples with targets at higher levels of abundance. However, all of these approaches involve target loss and are susceptible to sample matrix effects at high sample loading levels as well as outright failure due to sample overload. (Matrix effects are perturbations to the performance of a separation technology generated at high sample loading levels by the sample matrix participating as its own phase in the separation process rather than merely exchanging between the phases presented by the technology) While countercurrent chromatography and other dual liquid phase separation techniques support the fractionation of large quantities of biological sample with zero target loss they too are susceptible to phase collapse and sample matrix effects at the loading levels necessary, and they are intractable for processing analytical samples because sample runs are time intensive (many hours) and may not be pursued in the parallel high throughput manner that the economic feasibility dictates.

What is required is a separation process that is more robust to very high sample loading, has very low sample loss, achieves significant target enrichment and may be pursued in a parallel high throughput manner. To be robust to very high sample loading the system must anticipate matrix contributions to the phase partitioning as well as specific interactions between the matrix and the target and neutralize as much as possible both of these effects and the system must be wholly separable from the target. How to do this was not obvious. To achieve very low sample loss the system must employ liquid-liquid separation systems at the initial stages when high loading levels of sample requires very large system capacity relative to target abundance. Solid phase extraction may be implemented after dramatic target enrichment is achieved when system capacity need not exceed target abundance to the point where irreversible adsorption is appreciable.

The serial implementation of simple a dual solvent system followed by standard solid phase extraction technique would not produce adequate enrichment of low abundance targets. To achieve sufficient target enrichment it is necessary to leverage features of the target as specifically as possible. Physical separations generally exploit target polarity, antibodies recognize the structural features of the target. Employing the reactive functional groups to modify the target with the intent to perturb the target's behavior in a subsequent chromatography could in principal also be implemented to achieve sufficient enrichment. In particular if a separation process were applied to the sample removing all of the sample substituents that would normally populate a chromatographic fraction and then a covalent modification is applied to the sample to cause the target to now occupy the cleared chromatographic fraction if the separation process is reapplied, then it would be possible to achieve significant enrichment. The modifications would have to be specific enough to affect a tiny subset of the sample; produce a large enough perturbation to the target's behavior in the separation process to move the modified target into the cleared chromatographic fraction; be chemically feasible in the sample and separation milieu; and any remaining excess reactants could not confound subsequent analysis. How to do this was not obvious.

The present disclosure provides a method that addresses each of these four factors and enables the analytical quantification of oxytocin and vasopressin from, for example, whole blood, plasma, whole cow's milk, cow's cream, human breast milk and saliva by liquid chromatography tandem mass spectrometry to a limit of quantification of 20 pg/mL. One of skill in the art would understand that other biologicals may be used.

To generate a solvent system that leverages the sample matrix's contribution to the two phase solvent system; neutralizes both the matrix's interaction with the system and the interaction between the matrix and the target; is general enough to be used for most animal samples including those with significant cellular content; and create a system that is completely separable from the target; the samples are mixed with guandine hydrochloride (for example, to a concentration in excess of about 3 molar) and beta-mercaptoethanol is added (for example, to a concentration in excess of about 1 molar). In certain embodiments, the samples are mixed with guandine hydrochloride to a concentration in excess of about 0.5 molar, or about 1 molar, or about 2 molar, or about 3 molar, or about 4 molar, or about 5 molar, or about 6 molar, or about 8 molar, or about 10 molar or more.) In certain embodiments, the samples are mixed with beta-mercaptoethanol to a concentration in excess of about 0.1 molar, or about 0.2 molar, or about 0.5 molar, or about 1 molar, or about 2 molar, or about 3 molar, or about 4 molar, or about 5 molar, or about 6 molar, or about 8 molar, or about 10 molar or more.) That thiols (phosphine reducing agents did not enable this system) and in particular such a high concentration of thiol was required was not obvious.

The stabilized samples are extracted exhaustively with organic solvents to remove all the sample components that would normally fill ethyl acetate and acetonitrile partitions of plasma. Suitable organic solvents include but are not limited to ethyl acetate, acetonitrile, ethyl ether, etc. The guanidine and beta mercaptoethanol stabilized plasma is remarkably stable to extraction, for example tolerating up ten equal volume extractions (e.g., five with each solvent), without loss of phase performance. This is the first system able to performed this well on high protein content sample matrices like plasma and milk. In combination with the organic washes it also tolerates well cellular content from saliva and whole blood. By the second organic solvent (e.g., acetonitrile) extraction both phases are usually clear and colorless and there is no solid material at the phase interface.

Once the sample matrix has been cleansed of essentially all the components that are more hydrophobic than the targets, it is useful to modify the targets to make them more hydrophobic. If the modification is specific enough, the modified targets will then transfer into the next organic solvent (e.g., acetonitrile) extraction of the sample and little else will. Modifying a peptide in a complex aqueous mixture requires a robust chemical reaction. Modifying a peptide specifically means modifying the most unique feature or better set of features that may be found. Both oxytocin and vasopressin have amino-terminal cysteines that readily form thiazolidines in the presence of electron withdrawn aryl aldehydes. This reaction has been used to add chromophores to cysteines but this is the first report of modifying oxytocin and vasopressin as well as the first report of modifying the target for the purpose of affecting its chromatographic behavior. Being able to exploit the amine and the thiol of the cysteine at the first position of oxytocin and vasopressin gives the modification strategy selectivity for only those compounds with an amine and a thiol on vicinal carbons. This is not an abundant species in biological samples. And this reaction allows us to selectively modify the target in the presence of greater than 10 orders of magnitude excess beta mercaptoethanol thiol. In one embodiment of the methods, an electron withdrawn aldehydes may be used for the modification. In some embodiments, 4-phenoxy benzaldehyde is used. For example, 4-phenoxy benzaldehyde affords a facile reaction and significant chromatographic shift. After a reaction of the matrix with the aldehyde the reaction is quenched by adding an excess of cysteine (e.g., about two-fold excess, or about three-fold excess, or about four-fold excess, etc.). The cysteine thiazolidine is more polar than the aldehyde starting material as well as the target adducts and its formation improves the performance of the extract in the subsequent chromatography. The modified targets are extracted into an organic solvent (e.g., acetonitrile). The components of the system added to manage the matrix (guanidine hydrochloride and molar concentrations of thiol) are readily separated from the target at this step.

To insure both target and analytical system stability over large numbers of runs it is necessary to subject the organic solvent (e.g., acetonitrile) extract to a solid phase extraction. This extraction also enables another chemical modification of the target. Oxytocin and vasopressin both contain another thiol at internal cysteines. Alkylating that thiol with a hydrophobic reagent makes the target specifically even less polar than all the previously modified compounds. It also stabilizes the reactive thiol. Performing this reaction while the target and the modifying reagent are concentrated by being adsorbed onto the solid phase allows conducting this reaction with a minimal amount of reactant in a reasonable time. In one embodiment, benzyl 2-bromoacetate may be used. The excess alkylating reagent may be quenched with a cysteine chase, this enables sample storage and concentration. The doubly modified targets may then be washed of their co-extractants and eluted from the plate in a reproducable manner by gentle uniform positive pressure applied by identical columns of water under centrifugation. Eluted samples are clean enough for robust analysis (˜100 injections without intervention) on analytical instrumentation.

The present invention describes a novel rapid, high-throughput method for measuring oxytocin in small volumes of human plasma and serum. Volumes can be as small as 400 μL. Using the methods described, oxytocin can be detected in the plasma of healthy individuals on the order of 100-10,000 pg/mL. The invention discloses a novel method for processing a biological sample and obtaining an oxytocin and vasopressin enriched fraction such that they are more readily detected by analytical methods. To concentrate and further enrich the oxytocin and vasopressin within the enriched fraction for analysis and quantification, the method of the present invention employs a solid phase extraction reverse-phase C18 and a reverse phase C18 ultrapressure liquid chromatography with inline electrospray tandem mass spectrometry. The levels of oxytocin measured by this new method are 10-10,000 fold higher than the levels frequently reported with the two commercially based polyclonal antibody based methods. In addition, the oxytocin measured by this new method is 10,000 fold higher than reported in previous mass spectrometry assays. The method is also exhibits high sensitivity. Using the method of the invention, the oxytocin detection limit is 0.1 fmol. The invention discloses a method of detecting higher levels of oxytocin than can be detected by any other method, including mass spectrometry or polyclonal antibody assays.

The disclosure presents a model to explain the significantly higher levels of detected peripheral oxytocin, a model that allowed prediction of results that were observed while improving the preparation and isolation method of the invention. Biological fluids contain numerous compounds that are sequestered, frequently by proteins, away from the bulk of the solution. Oxygen is sequestered by hemoglobin, iron by transferrin, hepicidin is bound to a-2-macroglobulin, corticosteroids are bound by albumin, corticotrophin releasing-hormone is bound by the corticotrophin releasing-factor binding protein. This list is far from exhaustive. The biological activity of many things in the blood is spatio-temporally regulated by association with a protein that effectively sequesters the activity of the bound compound from the surrounding environment. Only upon encountering conditions where the association between the ligand and the sequestering protein is disrupted are the ligand and its biological activity released into the local environment.

Despite the pervasive use in nature of this strategy for providing precise local and temporal control for the biological activity of compounds in the blood, strategies for quantifying compounds with biological activity only infrequently attempt to disrupt the sequestering interaction prior to quantifying the plasma load of a compound with biological activity. While there have been attempts to use denaturant to disrupt sequestering interactions this patent discloses the first attempt to perturb the redox state of the biological fluid to disrupt a sequestration. The reducing compounds, like low molecular weight thiols and compounds like histamine that can catalyze oxidation by stabilizing electron rich metal ions, are used. Without being bound to a particular theory, it is believed that perturbing the redox state of a biological sample will be particularly useful for generating fractions enriched with thiol containing peptides that may be sequestered through mechanisms of disulfide exchange.

Oxytocin and vasopressin undergo disulfide exchange with abundant thiol containing compounds under aerobic conditions like those found in blood plasma. When exposed to reduced glutathione (1 mM) and oxygen at their blood concentrations oxytocin (˜10 nM) and vasopressin (˜10 nM) form diglutathione adducts. In the presence of reduced glutathione and reduced cysteine (0.5 mM) and oxygen, oxytocin and vasopressin form dicysteine adducts. If albumin (1 mM) is added to that mixture all detectable oxytocin and vasopressin sequester with albumin upon acetone precipitation. Oxytocin immunoreactivity in plasma is associated with high molecular weight species (Szeto et al). When beta-mercaptoethanol (1 M) and histamine (30 mM) are added to plasma, oxytocin and vasopressin in the 1-100 nM range were recovered rather than the 1-100 pM range recovered without thiols or reported routinely with existing commercially available immunoreactivity tests. The mid nanomolar range is much more consistent with the concentrations required in biological assays for these compounds, such as inducing the lactation reflect with rabbit mammary tissue. Nanomolar concentrations for oxytocin and vasopressin are also far more consistent with the doses required for either peptide in intervention regimes.

Disulfide exchange has been used to purify compounds from plasma. Thiol functional groups are used to terminally functionalize solid support matrices and contacted with plasma under native conditions. It is possible in this way to isolate peptides with exposed free thiols and to elute them specifically with the addition of excess specific thiol. This strategy could be used in principle for a thiol chemically protected in a disulfide bond. However, for a compound sequestered from bulk plasma, possibly even sequestered in part by a disulfide bond this approach will be of limited utility. Because disulfide exchange is fast relative to most protein dissociation kinetics the low effective molarity of resin constrained thiols will be insufficient to dominate the exchange with the native disulfide partners even under conditions of dramatic denaturation. Further the more denaturant required the lower the specificity of the procedure. Redox sequestered compounds are released by perturbing the redox state of the plasma with a vast excess of low molecular weight thiols like beta mercaptoethanol or dithiothreitol and imidazole containing compounds like histamine, histidine and imidazole.

In one aspect, the invention describes a method of processing a biological sample prior to the quantification of the molecule of interest. This method employs two distinct steps that will be key to the quantification of several different types of molecules derived from biological samples moving forward.

In a first aspect of the invention, the disclosed method involves contacting the biological sample containing the relevant biological molecules with compounds that perturb the redox state of the sample (e.g., thiols or phosphines and imidazoles and peroxides). Perturbing the redox state will, among other effects, release redox sequestered compounds and facilitate their detection. Failing to release redox sequestered compounds from biological samples will in some cases lead to failure to isolate a considerable portion of the target molecules in question. If the target molecule is sequestered by the redox state of the system and the sequestered compound partitions in a way different from the free compound in the applied chromatography, then a portion of the target molecule present in the sample will be overlooked. Furthermore, target sequestration dependent on the redox state of the system is highly mutable under biological conditions. Therefore, only by obtaining the total load of the redox sequestered (but readily released) compound one of skill in the art will be able to accurately anticipate effect of the target on the sample.

In one embodiment of the invention, the redox modifiers are reducing agents, oxidizing agents, denaturants and detergents. In another embodiment of the invention, the redox modifiers are reducing agents and oxidizing agents. In one embodiment of the invention, the redox modifiers are thiols, phosphines, imidazoles and peroxides. In one embodiment of the invention, the redox modifiers are thiols and imidazoles. In one embodiment of the invention, the redox modifiers are beta mercaptoethanol and histamine. In another embodiment, the biological sample is subjected to a high concentration (relative to blood) of beta mercaptoethanol and histamine. In certain embodiments, beta mercaptoethanol concentration is at least 100 mM and the histamine is at least 10 mM. In a preferred embodiment, the beta mercaptoethanol concentration is 1M and the histamine concentration is 60 mM.

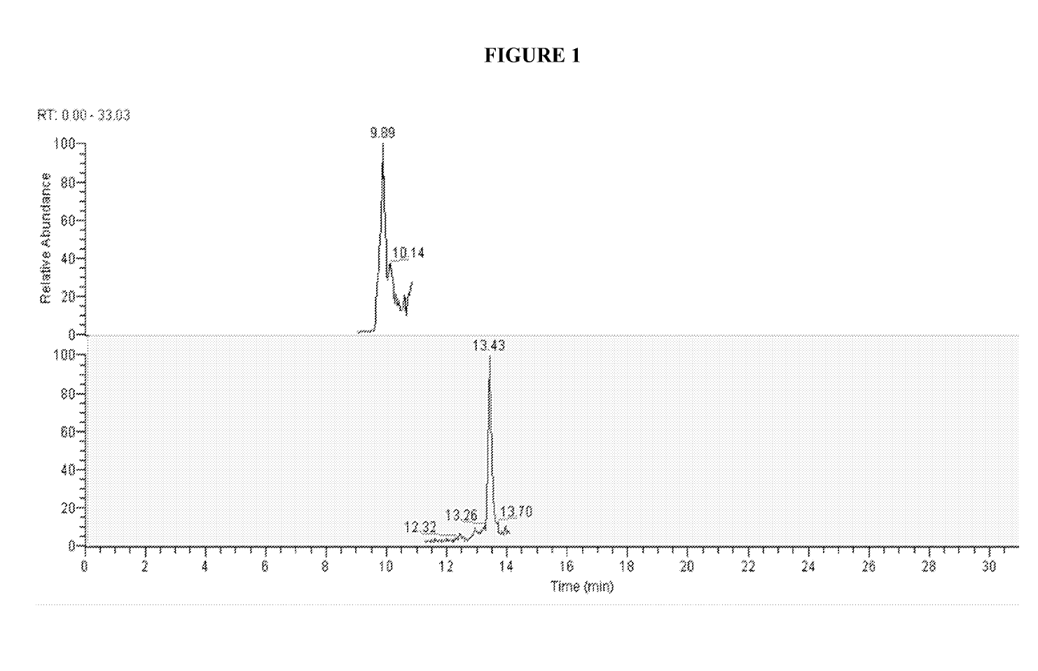

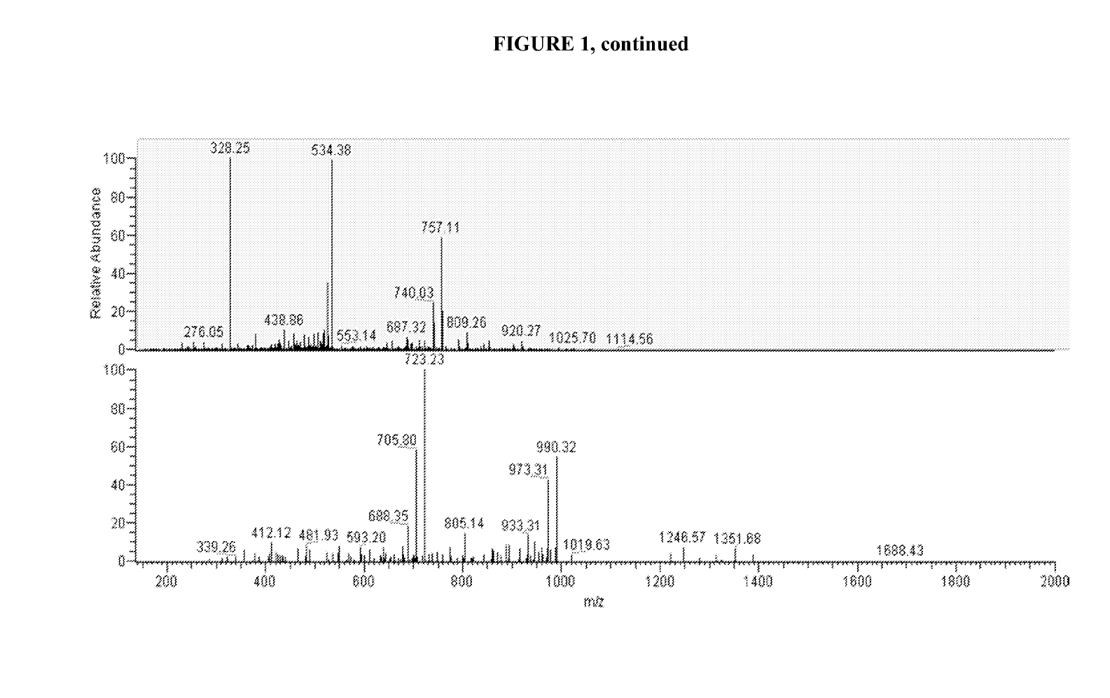

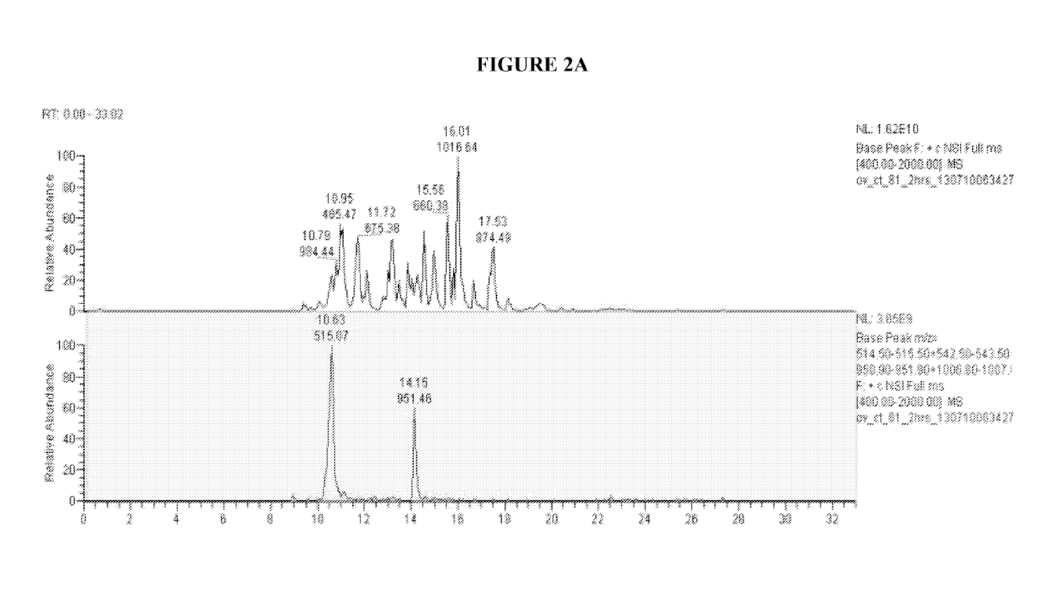

In certain embodiments, the biological sample is contacted by a reducing agent and by an oxidizing agent. In other embodiments, only the reducing agent is present. In other embodiments, only the oxidizing agent is present. The effect of beta mercaptoethanol and histamine independently and collectively on the recovery of oxytocin and vasopressin from plasma can be measured. To do so, the biological sample was contacted by beta mercaptoethanol and histamine, beta mercaptoethanol alone, histamine alone, and water only. The volume of buffer removed was replaced with water. The recovery of oxytocin and vasopressin without both beta mercaptoethanol and histamine was < 1/10 of the total obtained with both present. The recovery with only beta mercaptoethanol added was 1/10 the total obtained and the recovery with only histamine added was 1/10 the total obtained with both present. Each component alone will enhance the detection of oxytocin, but the combination of both components produces the best detection. These results are presented in FIG. 1.

In one embodiment, the denaturants and detergents are applied in gradient observations with slow alkylating phosphine reducing agents added in excess. With the plasma normalized, the proteins denatured and the thiols reduced, the amino-terminal cysteine may be coupled with a benzaldehyde derivative at alkaline pH. Iodoacetamide may then be used to alkylate the second thiol in oxytocin. This resultant mixture may be subjected to a variety of bulk fractionation and solid phase extraction processes to obtain the combination that is compatible with oxytocin isolation from the initial mixture.

In one embodiment, the 4G11 monoclonal antibody may be anchored 4G11 directly to NHS-activated Sepharose. If the immunoreactivity profile of 4G11 has subnanomolar dissociation constants for all forms of oxytocin and if the solid support shows subfemtomole non-specific adsorption then 4G11 will be suitable for use in oxytocin fraction generation. In one embodiment, the ultra-filtration and dialysis may be compared with organic, acid, and salt-based precipitations. Solid-phase extraction protocols may evaluate reverse-phase, ion-exchange and mixed-mode matrices with varied organic and pH elution regimes.

In a second aspect of the invention, the disclosed method involves fractionation of the biological sample using precipitation. After the redox sequestered compounds are released from their binding components, the binding components and other high molecular weight proteins may be removed from the solution by precipitation. Any organic solvent that can precipitate high molecular weight molecules while retaining the molecules of interest dissolved in solution can be used. In certain embodiments, a solvent that separates other larger molecules from the target molecules in the sample is used. In one preferred embodiment the solvent used is acetonitrile (2 volumes). In one preferred embodiment, the solvent used is acetone (4 volumes). Oxytocin and vasopressin, oxytocin and vasopressin diglutathione and dicysteine will all remain in solution while larger proteins and peptides will precipitate.

In one embodiment, the biological sample is human plasma, from which oxytocin and vasopressin can be isolated and purified. Plasma is a complex sample with high molecular weight compounds, mostly proteins, present at very high concentrations. For example, the concentration of albumin may be as high as 700 μM. Removing high molecular weight components from plasma is critical, so that low molecular weight constituents like oxytocin and vasopressin can be detected. The precipitation step is performed with acetone. The biological sample containing the molecule of interest is diluted with acetone, with the intent of precipitating the abundant large proteins.

In a third aspect of the invention, the disclosed method involves further purification and concentration of the oxytocin and vasopressin enriched fraction—the supernatent from the acetone precipitation of the redox perturbed biological sample—by first allowing evaporation of the organic solvent and second by submitting the supernatent to solid phase extraction with reverse phase C18 silica. This step removes the majority of the low molecular weight components of the blood from the supernatent, including the most hydrophobic lipids and allows concentration of the desired compounds better enabling their detection. Failure to pursue this step reduces the assay sensitivity and reproducibility and limits the lifetime of the downstream columns.

In one embodiment, only the organic solvent is evaporated under (atmospheric pressure) from the supernatent and the aqueous portion is applied to a Seppak tC18 uElution plate. In this embodiment the sample is applied to the resin under binding conditions, washed and eluted under non-binding conditions. In one embodiment the binding conditions are aqueous buffered at pH 7.4 and 20 mM betamercaptoethanol, the wash conditions are 10% acetonitrile buffered at pH 7.4 and containing 20 mM betamercaptoethanol, and the elution conditions are 20-30% acetonitrile buffered at pH 7.4 or 30% acetonitrile buffered at pH 2.0.

In one embodiment that is broadly applicable to a number of targets, a decoy that is as much like the target as possible but distinguishable by mass is added to the sample to allows the elution of target off the sample (when it is bound) without using detergents that confound downstream purification. For example, the process is useful for the detection of insulin, estrogen, cholesterols, oxytocin and vasopressin.

In one example directed to oxytocin and vasopressin, these target analogs may be the same peptides without the C-terminal glycines: the synthesized oxytocin analog would be CYIQNCPL (SEQ ID: 6), vasopressin analog CYFQNCPR (SEQ ID: 7). As an alternative, a chymotrypsin digest of oxytocin and a trypsin digest of vasopressin can be used. The tyrosine in oxytocin is shielded from proteolysis by the disulfide bond.

In one embodiment, two isotopically labeled oxytocin (9) and oxytocin-gkr (12) peptides may be employed. An oxytocin+7 amu and an oxytocin+14 amu peptide, as well as an oxytocin-gkr+7 amu and an oxytocin-gkr+14 amu peptide are used. The isotopic labels in these peptides are achieved with leucine and isoleucine amino acids containing six ¹³C atoms and one ¹⁵N atom. ¹³C and ¹⁵N atoms interact with chromatographic resins exactly the same way as ¹²C and ¹⁴N the naturally most abundant nuclei. The isotopically labeled peptides elute at exactly the same time as the naturally occurring peptide, with the same peak shape in the same proportions in the same ionization droplets generating perfectly matching ionization efficiencies. This is in contrast to the more common but the less precise approach using deuterium (²H) atoms that adhere to column matrices more than hydrogen atoms (¹H), and elute later in separate droplets, ionizing with different efficiencies, generating the need for interpreted quantification. In one embodiment, the two peptides are independently employed in every experiment. For example, the oxytocin+7 and oxytocin-gkr+7 peptides are added into the plasma at a known concentration upon thawing the plasma. The oxytocin+14 and oxytocin-gkr+14 peptides are added to the elution from the final pre UPLC step in the analysis. The +14 peptides provides precise validation of each sample quantity loaded and the efficiency of the ionization precisely when natural oxytocin ionizes, the +7 peptide reports the efficiency (within each experiment) of the oxytocin enrichment procedure. The amount of the labeled and unlabeled naturally occurring peptide detected is quantified. By quantifying the amount loaded in the measurement experiment and the efficiency of the measurement experiment it is possible to give the most confident quantification incorporating in sample measurement of experimental error rather than average values. This approach quantifies experimental error, instrument error and procedure failure in every single sample quantified.

### EXAMPLE

The compositions and methods of the disclosure are illustrated further by the following examples, which are not to be construed as limiting the disclosure in scope or spirit to the specific procedures and in them.

### Materials and Equipment

Nano LCMSMS Capability:

Waters nanoAcquity ultra performance liquid chromatography system in-line nanospray ionization, and a Thermo Deca XP+ ion trap mass spectrometer. The nano LC-MS/MS setup is capable of robust peptide identification from samples containing 100 fmols of material.

Software Tools for Analyzing Mass Spectra:

Discoverer 1.0 (replaced Sequest) peptide and protein assignment software.

Protein Modification Reagent Synthesis and Purification:

Cold water plumbed chemical fume hood, Buchi R114 rotary evaporator with Welch pump, Mettler Toledo AT261 Delta Range analytical balance, Foxy 200 flash chromatography system, glassware oven, Chemglass glassware and 4 C, −20 C, −80 C storage.

Protein Structural Biology:

Complete structural biology work station with 0, CNS, and CCP4, software suites installed as well as a variety of other structural biology software tools.

Protein Biochemistry and Molecular Biology:

Elga PureLab Prima & Ultra 18 MOhm water purifier, Mettler Toledo PB3002-S/Fact balance, Accumet 25 pH conductivity meter, Beckman 530 UV/Vis spectrophotometer, two Eppendorf 5415D bench top centrifuges, Beckman Coulter Allegra 6KR clinical centrifuge, New Brunswick Scientific Innova 4200 incubator/shaker, MJResearch PTC-200 DNA engine thermocycler, BioRad E. coli pulser, and protein and DNA electrophoresis equipment.

### Example 1

### Obtaining a Thiol-Sequestered Compound (e.g., Oxytocin and Vasopressin) Enriched Fraction from Plasma

To obtain a redox-sequestered compound enriched fraction from plasma, dilute 400 μL freshly thawed plasma with 80 μL 300 mM histamine and 27.5 μL of neat beta-mercaptoethanol and mix. Incubate the mixture at room temperature for 20 minutes. Precipitate the high-molecular weight proteins with either 1.6 mL acetone or 800 μL acetonitrile and remove the precipitate by centrifugation for 1 minute at 16 kg at room temperature in an Eppendorf 5415D centrifuge. Remove and store the supernatent, allowing the organic solvent to evaporate off.

Wet a Seppak tC18 uElution plate with 700 μL 30% Acetonitrile in dioxane. Accelerate the solvent through the resin at 1000 rpm in an Allegra 6KR centrifuge with a G 3.8 rotor for 5 minutes at 4° C. Repeat wash once at 1000 rpm then once at 250 rpm. Add 700 μL acetonitrile buffered with 1 mM NaₓH_{y}PO₄ pH 7.4, accelerate through the resin at 250 rpm as above, and repeat once. Add 700 μL water buffered with 1 mM NaₓH_{y}PO₄ pH 7.4, accelerate through the resin at 250 rpm as above, and repeat once.

Add aqueous remainders from organic precipitates to the Seppak tC18 uElution plate, fill to 700 μL with 1 mM NaₓH_{y}PO₄ pH 7.4, accelerate through the resin at 250 rpm in an Allegra 6KR centrifuge with a G 3.8 rotor for 5 minutes at 4° C. Remove any remaining liquid (up to ˜50 μL, record the volume) from above the resin. Add 100 μL 1 mM NaₓH_{y}PO₄ pH 7.4, accelerate through the resin at 250 rpm for 5 seconds as above, repeat with 10% acetonitrile 1 mM NaₓH_{y}PO₄ pH 7.4. Elute oxytocin and vasopressin with 100 μL 20% acetonitrile in 1 mM NaₓH_{y}PO₄ pH 7.4 accelerate through the resin into a collection plate at 250 rpm for 5 seconds as above, 100 μL 30% acetonitrile in 1 mM NaₓH_{y}PO₄ pH 7.4 as above, and vasopressin with 100 μL 30% acetonitrile in 0.1% formic acid as above.

The oxytocin and vasopressin are further purified using column chromatography. The Seppak elutes are injected onto a trapping column (nanoACQUITY UPLC column, Symmetry® C₁₈, 5 μm, 180 μm×20 mm; Waters) and washed for 3 minutes with 98% 0.1% formic acid in water at a flow rate of 15 μl/min. The oxytocin and vasopressin are then separated on a nanoACQUITY UPLC column, BEH130 C₁₈, 1.7 μm, 75 μm×150 mm, Waters. Peptides were eluted from the column in a linear gradient for 18 minutes from 2 to 62% acetonitrile with 0.1% formic acid). The flow rate elution was set to 0.6 μl/min, and the analytical column was heated to 50° C. The oxytocin is then ready for analysis by liquid chromatography-quadrupole mass spectrometry (LCMS/MS).

### Mass Spectrometry of Isolated Oxytocin and Vasopressin

The UPLC column elute undergoes electrospray ionization at 1.75 kV through an 360 μm OD, 75 μm ID, 15 μm tip ID uncoated silica electrospray tip (New Objective). The ionized corona enters a ThermoFinnigan Deca XP plus LCQ ion-trap mass spectrometer through the capillary tube in the atmosphere pressure ionization stack. From 9 to 11 in the UPLC gradient when vasopressin elutes, and From 13 to 15 minutes in the UPLC gradient, when oxytocin elutes, the instrument duty cycle includes a single ion monitoring MS scan and a full MS/MS scan. The following instrument settings were used: number of scan events 2, single ion monitoring window (vasopressin at +2 542.4-543.3 m/z, oxytocin at +1 1006.8-1007.8 m/z), full MS/MS scan of the parent ion if the intensity is <5×10̂5, precursor isolation width 1, normalized collision energy 35%. Tuning parameters were as follows: capillary temperature 150° C., no sheath gas flow, no sweep gas flow, automatic gain control (AGC) was on, source voltage 1.75. The mass spectra data for the protein mixtures was collected during a total run time of 33 minutes.

### Example 2

### Oxytocin and Vasopressin in Plasma are Sequestered by Reversible Disulfide Exchange

A decoy that is as much like the target as possible but distinguishable by mass is added to the sample to allows the elution of target off the sample (when it is bound) without using detergents that confound downstream purification. Oxytocin and vasopressin target analogs may be the same peptides without the C-terminal glycines: the synthesized oxytocin analog would be CYIQNCPL (SEQ ID: 6), vasopressin analog CYFQNCPR (SEQ ID: 7). As an alternative, a chymotrypsin digest of oxytocin and a trypsin digest of vasopressin may be used. The tyrosine in oxytocin is shielded from proteolysis by the disulfide bond.

The following reagents and process are employed: 400 μL plasma (edta tubes, freshly thawed), 800 μL H₂O, 12 μL 1M NaHCO₃, 12 μL 1M Ascorbic Acid (fresh), and RT 20 minutes. To this was added: decoy to 1 μM (or not), 48 μL 100 mM Glutathione (fresh) 120 IA 300 mM Histamine (fresh) 60 uL neat beta mercaptoethanol, and RT until partially cloudy (i.e., less than 5 mins). Then, 100 μL 50% trichloroacetic acid was added, and pelleted 2′ RT Eppendorf 5415D centrifuge. Solid phase extraction of the supernatant is performed and the elution of the target off of C18 is done. LC-MS/MS analysis is conducted.

If the target levels are the same with and without the decoy the target elution plasma treatment regime of the plasma is optimal. If target levels are enhanced in the presence of the decoy, the target elution plasma treatment regime is not optimal.

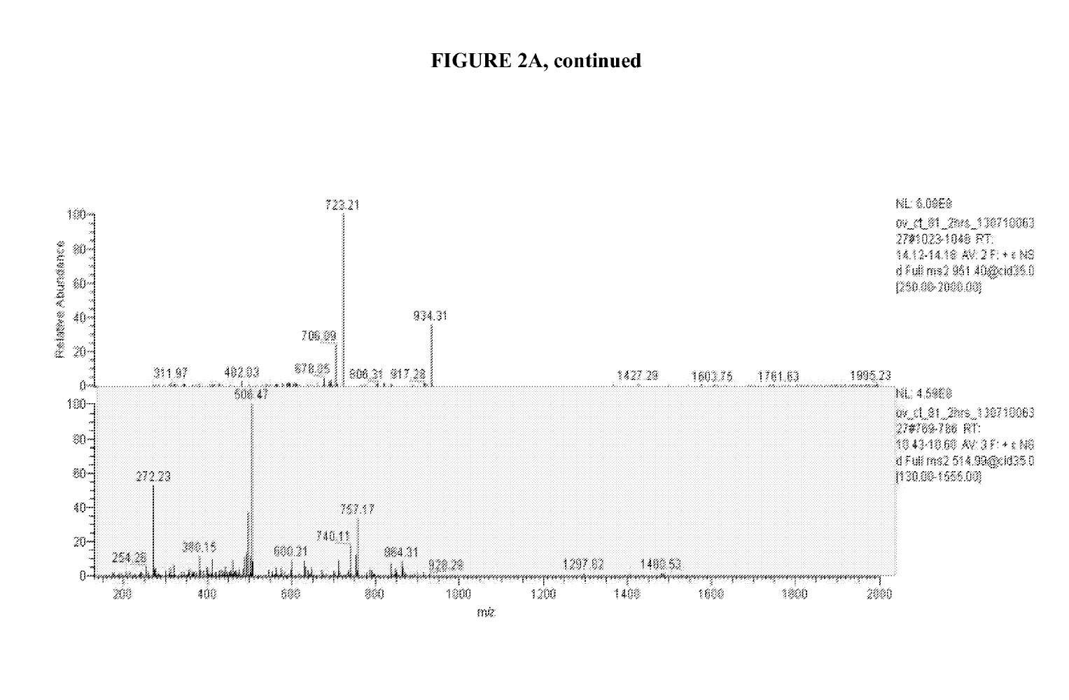

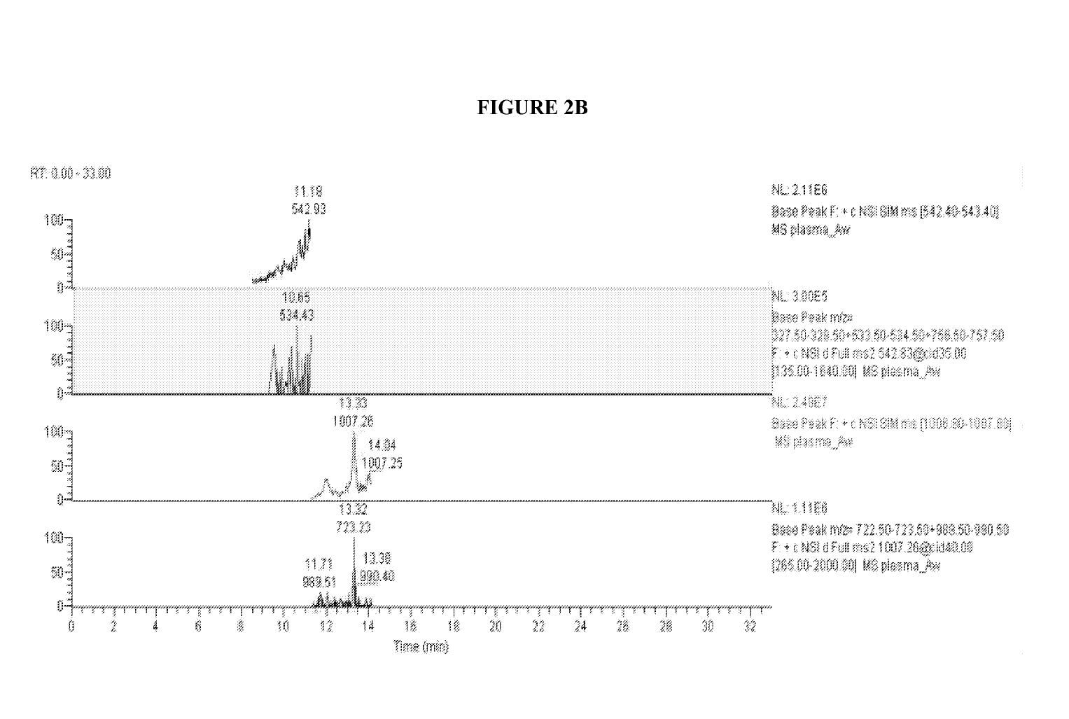

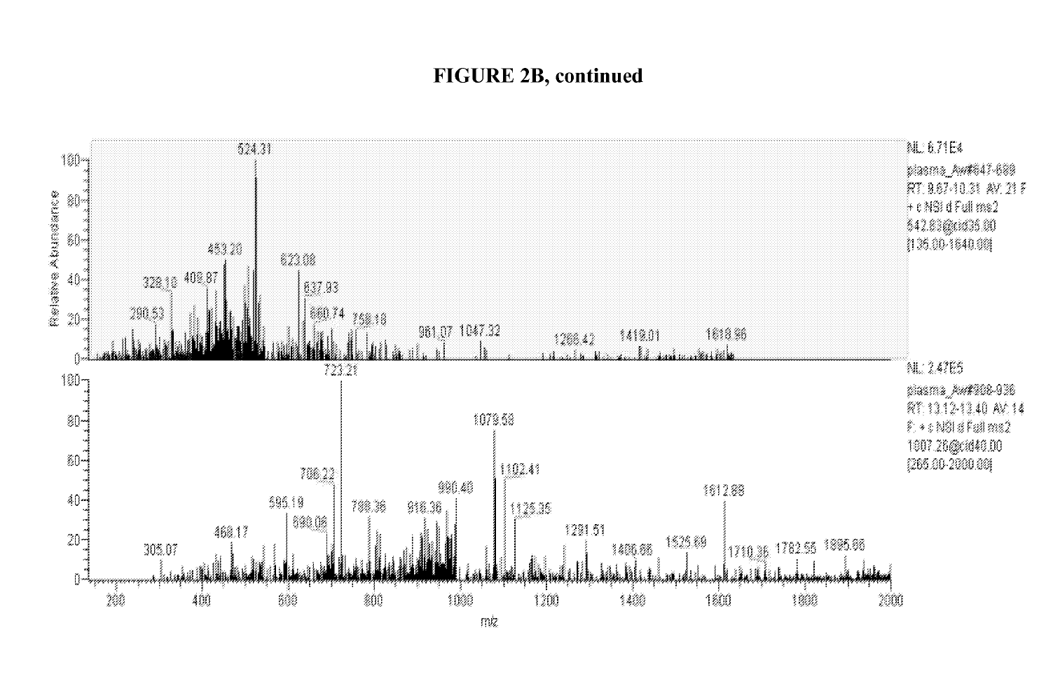

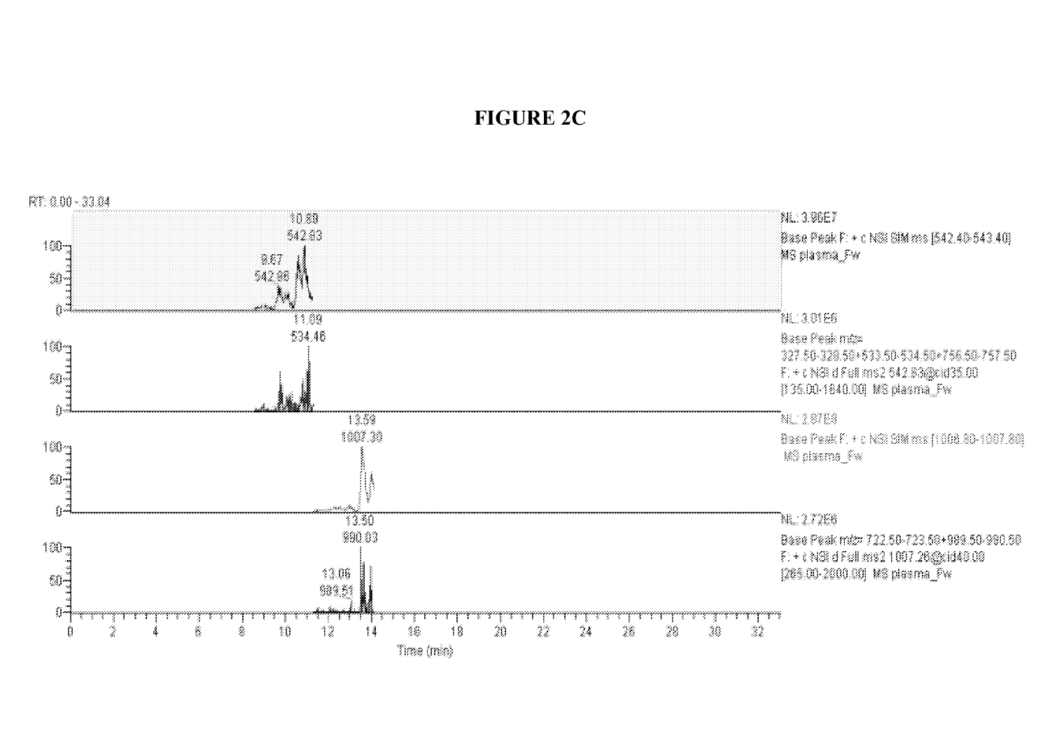

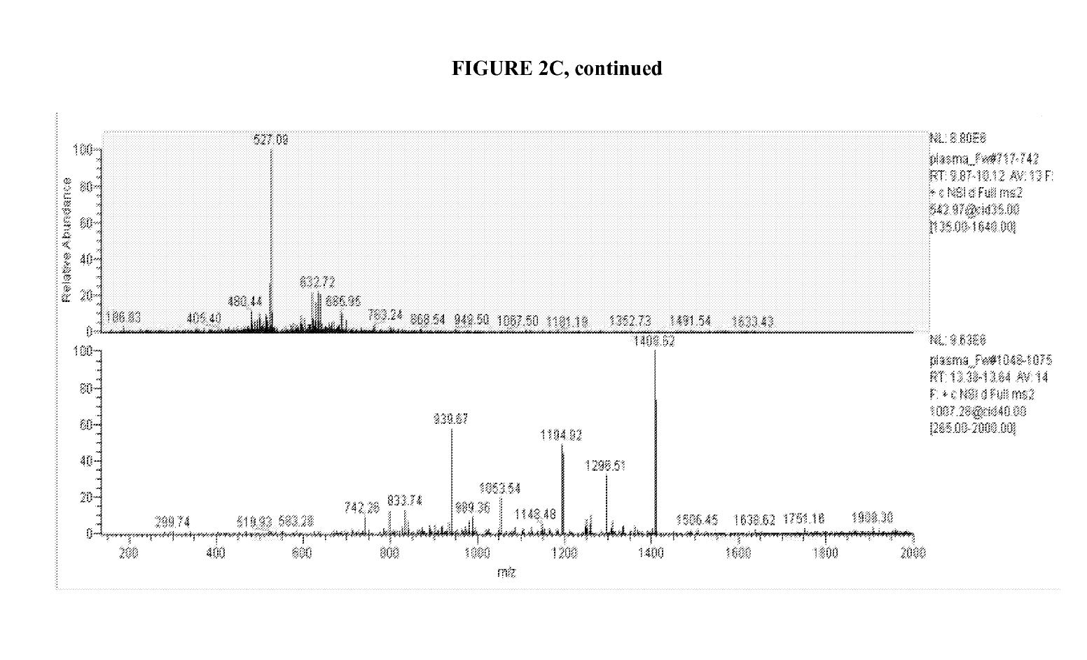

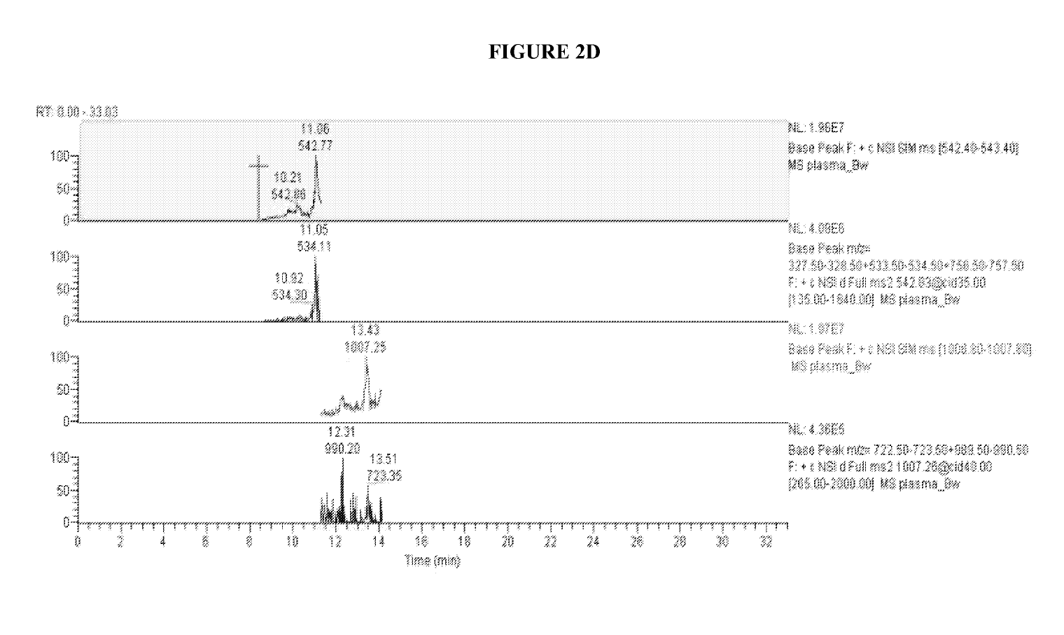

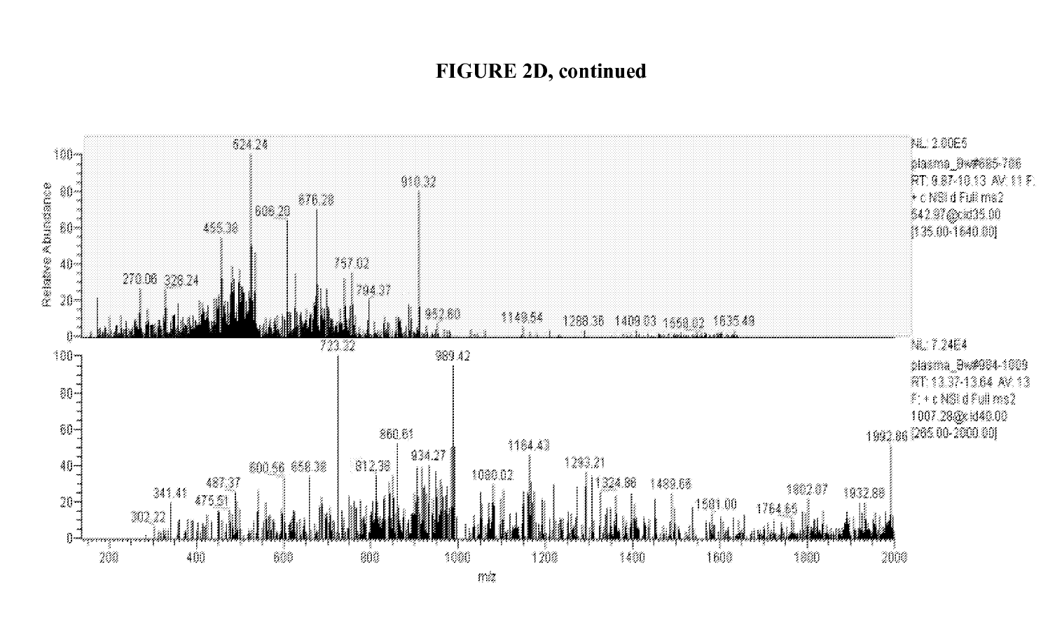

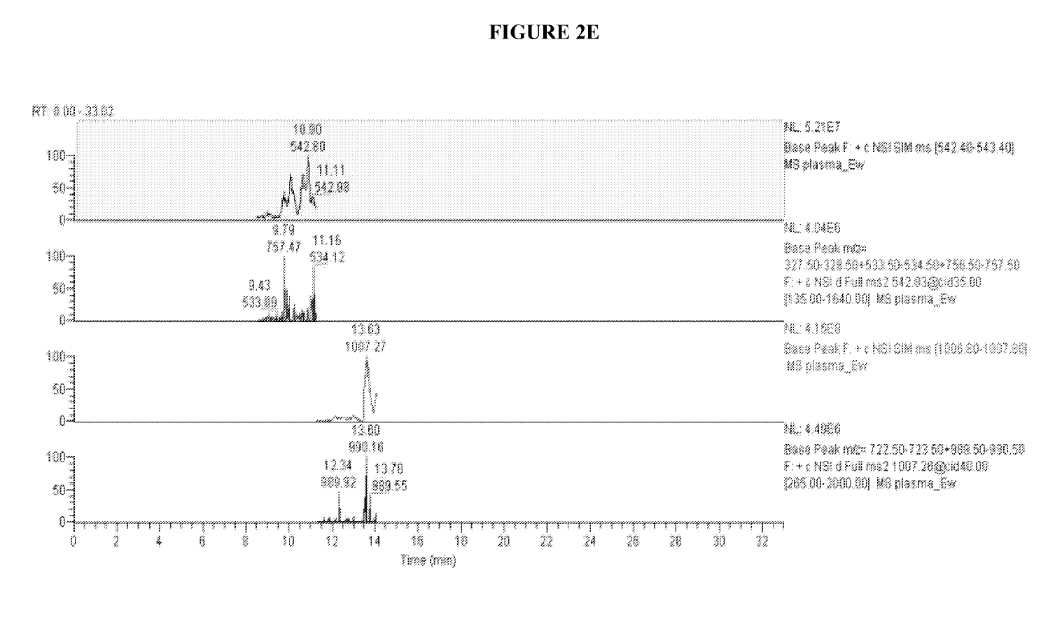

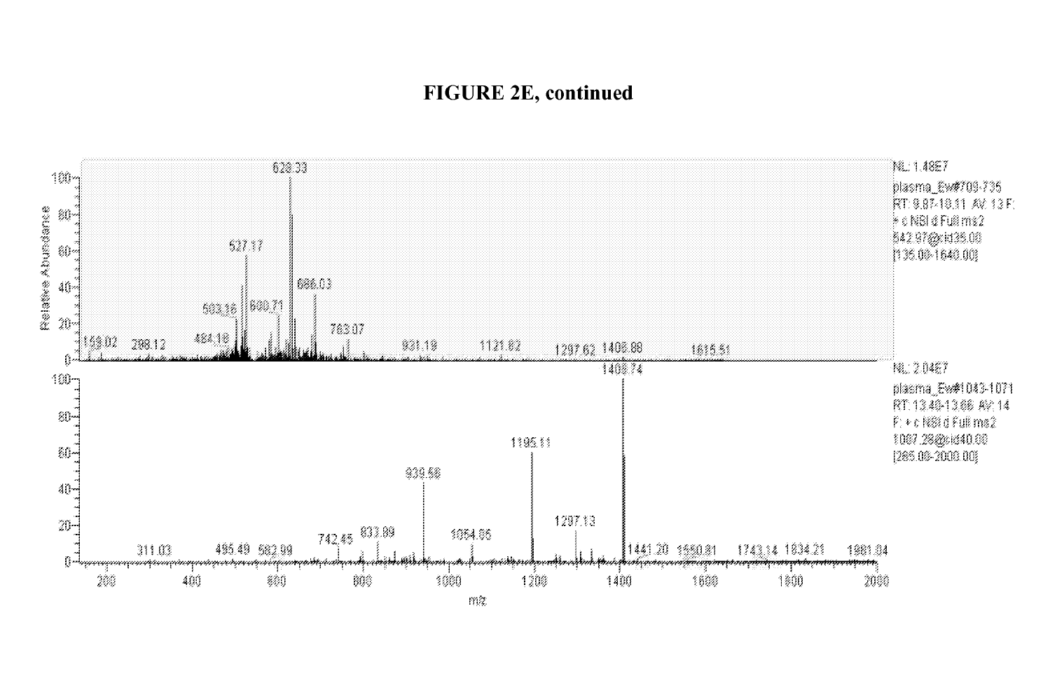

FIGS. 2A-E images show the results of MS of the analog peptides, plasma with the addition of the analogs, plasma in which has subject to the foregoing process, the decoy peptide, and buffer blank and another run showing the how the proteolysis generated the correct decoys. For oxytocin and vasopressin the decoys can be used with the above described redox process to break the disulfide bonds. The foregoing process involves a general strategy that is widely application to mass spec analysis of other peptides. In general, the method takes into account the protein bound portions of compounds when measuring by mass spec.

### Example 3

### Oxytocin and Vasopressin in Plasma are Sequestered by Reversible Disulfide Exchange

Measuring Oxytocin and Vasopressin in Plasma Using Liquid Chromatography Tandem Mass Spectrometry (LCMSMS):

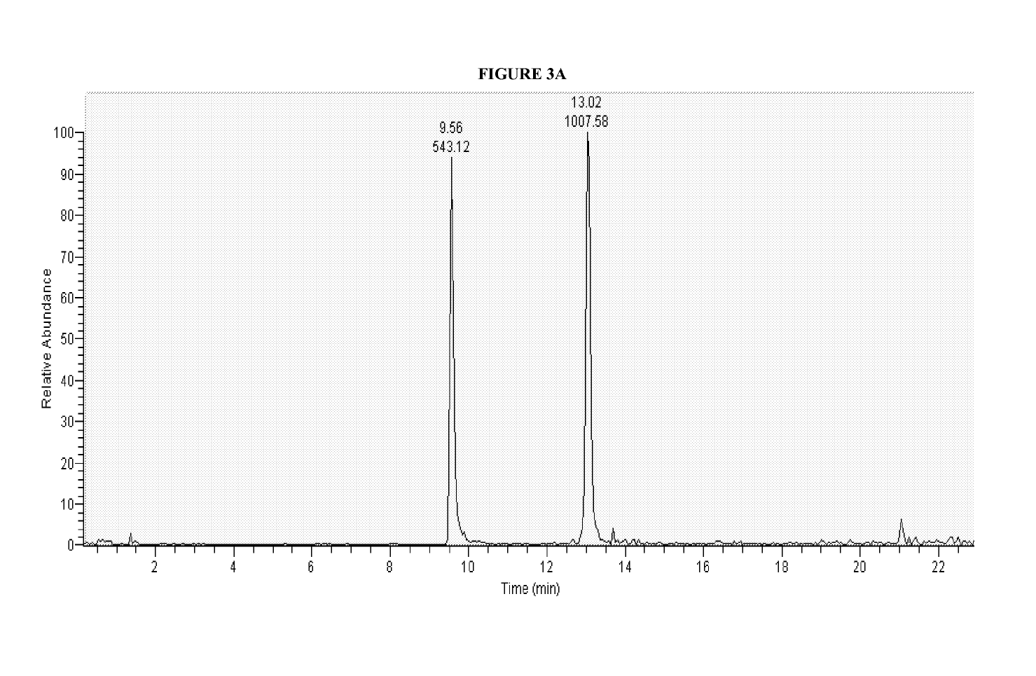

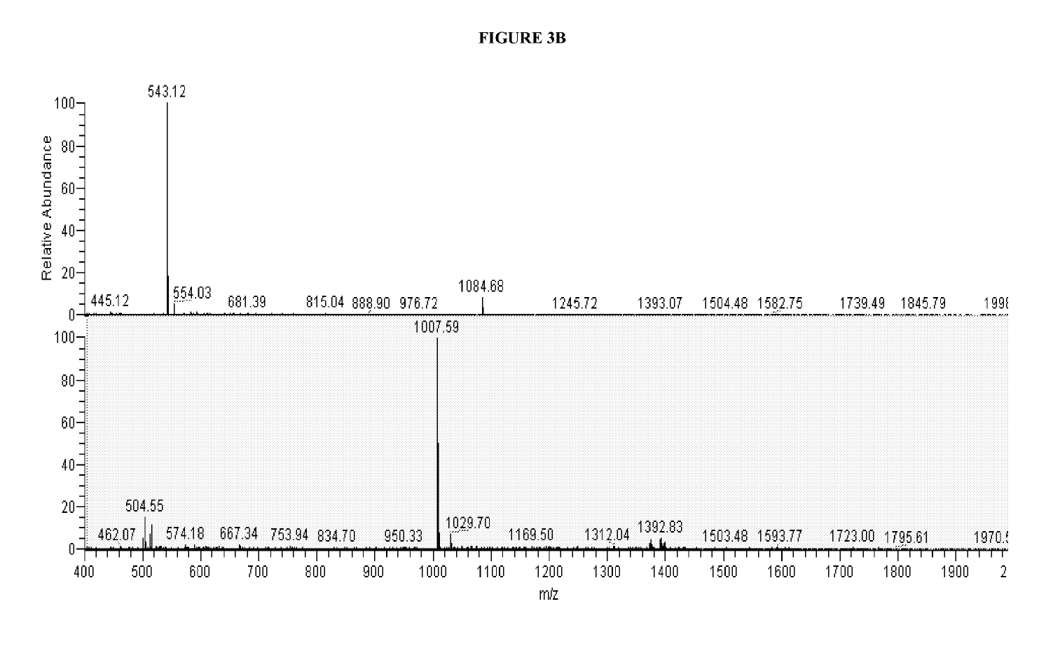

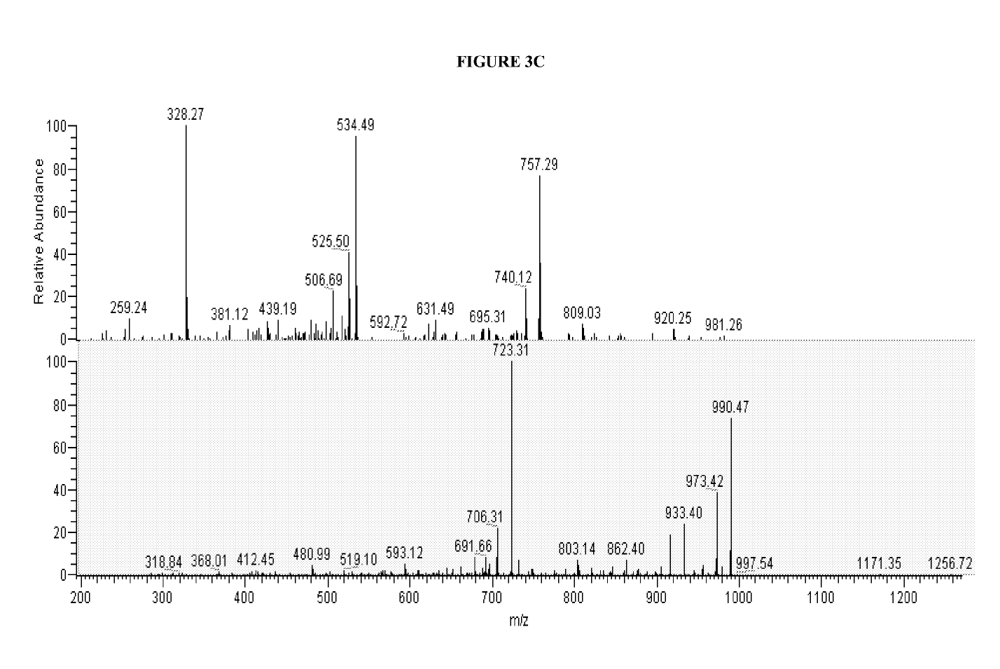

LC-MS/MS verifies the identity of the compound being quantified in every measurement (FIGS. 3A-C). The verification and quantification are informed by not by one number, but a retention time, a molecular ion mass and a fragmentation pattern of that ion that forms a compound fingerprint. LC-MS/MS is singular in its ability to achieve assignment and quantification of each compound in every measurement from crude mixtures like plasma and saliva fractions.

Oxytocin and Vasopressin Undergo Disulfide Exchange with Abundant Thiol-Containing Plasma Peptides:

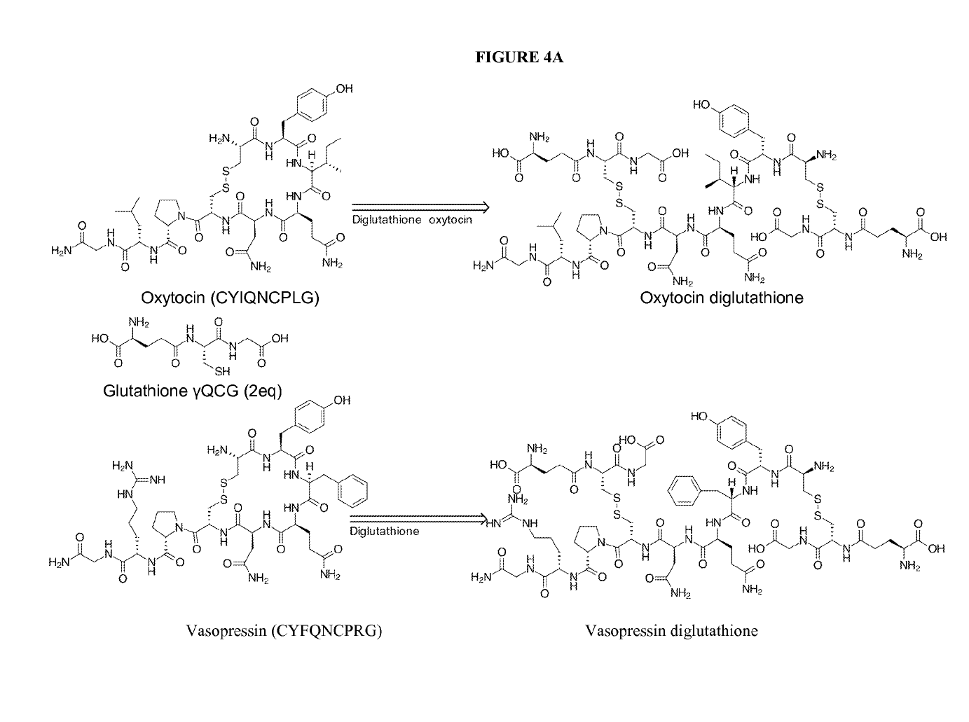

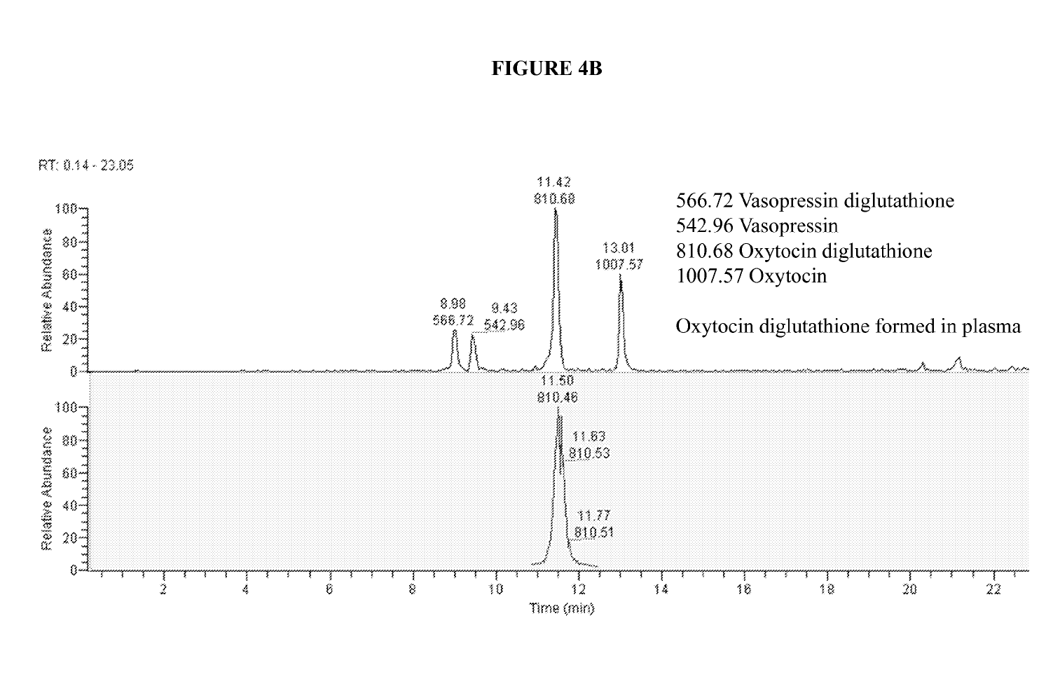

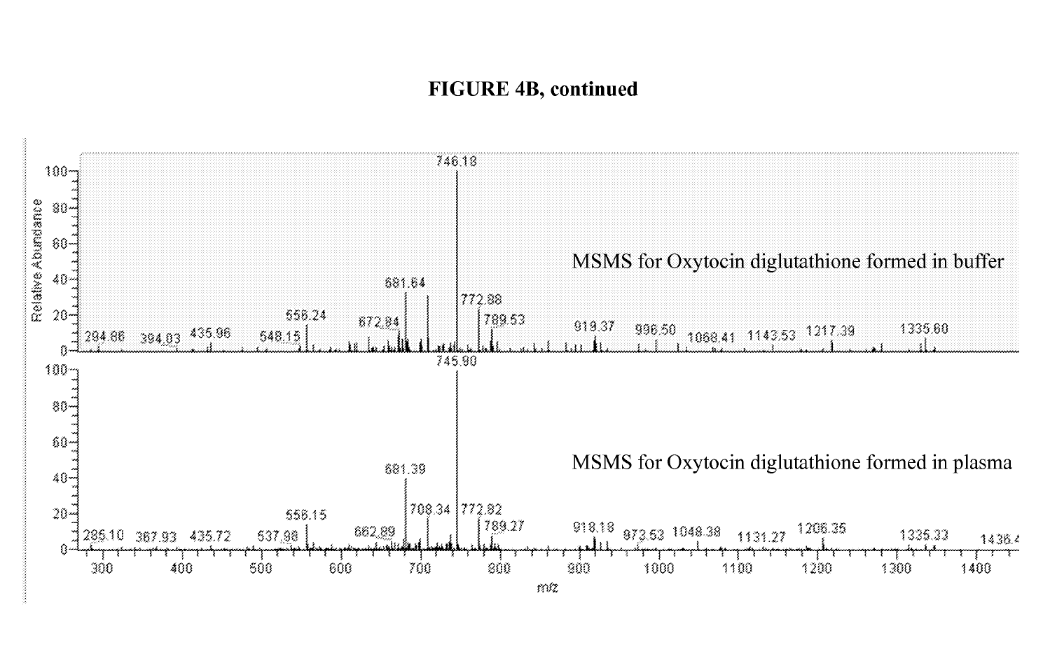

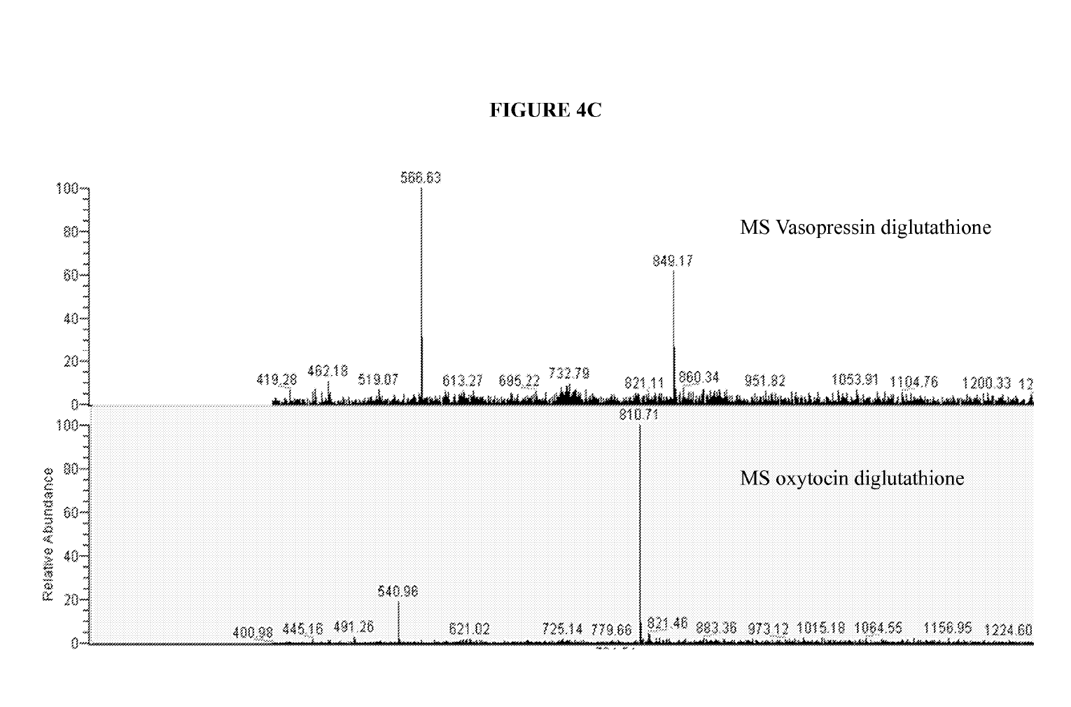

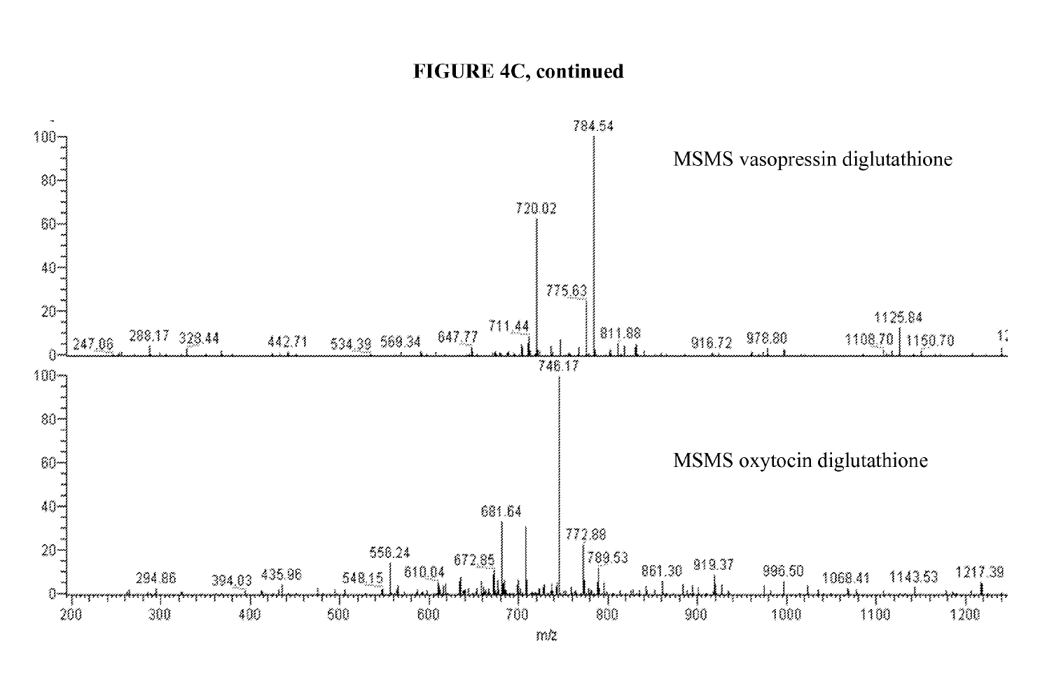

Incubating oxytocin and vasopressin them with glutathione at its plasma concentration in the presence of air (as in blood) induces oxytocin and vasopressin to disulfide exchange with glutathione and undergo additional oxidation resulting in the accumulation of diglutathione adducts (FIGS. 4A-C). This additional oxidation-containing product accumulates even in the presence of a vast excess of reducing equivalents in the form of remaining excess reduced glutathione. The introduction of air lead to a disulfide exchange result distinct from that observed in the published literature. The disulfide exchange is inhibited by EDTA which is added to most blood as a preservative. In the body the hormones undergo exchange, but in collected blood the EDTA will inhibit diglutathione formation unless the redox state of the collected blood is perturbed, as FIG. 5.

Oxytocin and Vasopressin Precipitate with Abundant Free Thiol Containing Plasma Proteins:

Incubating oxytocin and vasopressin with the free thiol containing albumin at pH 7.4, at room temperature (or 37° C.) with or without glutathione and cysteine leads to quantitative adsorption to albumin (FIG. 5). Acetone extraction of the incubation clears oxytocin and vasopressin from the solution into the pellet with the albumin. In one embodiment, exceptionally short half-life of oxytocin is measured with immunoreactivity on acetone extracted fractions. Observation of disulfide exchange mediated accumulation of oxytocin on high molecular weight proteins in the plasma.

Disulfide exchange with glutathione, and also with cysteine (not shown) adsorption/disulfide coupling to albumin or to the cysteine knot domain of von Willebrand's factor (implicated in vasopressin biology) and others are sufficient mechanisms to explain the ensemble of immunoreactive fractions observed in Szeto et al. (2011). As the closed disulfide ring form of oxytocin and vasopressin are the biologically active forms, it is understood that disulfide exchange creates an ensemble of inactive hormones in plasma that are sequestered, in different forms that may have distinct biological meanings. Further, because disulfide exchange is reversible, exceptionally fast and modulated broadly within physiological conditions it provides a powerful local switch for changing the biologically active local concentration of oxytocin and vasopressin. This switch would be flipped in inflammation, at a wound and likely at any alteration to the integrity of the vascular endothelium.

Exogenous Oxytocin and Vasopressin May be Eluted from Acetone Extraction Pellets:

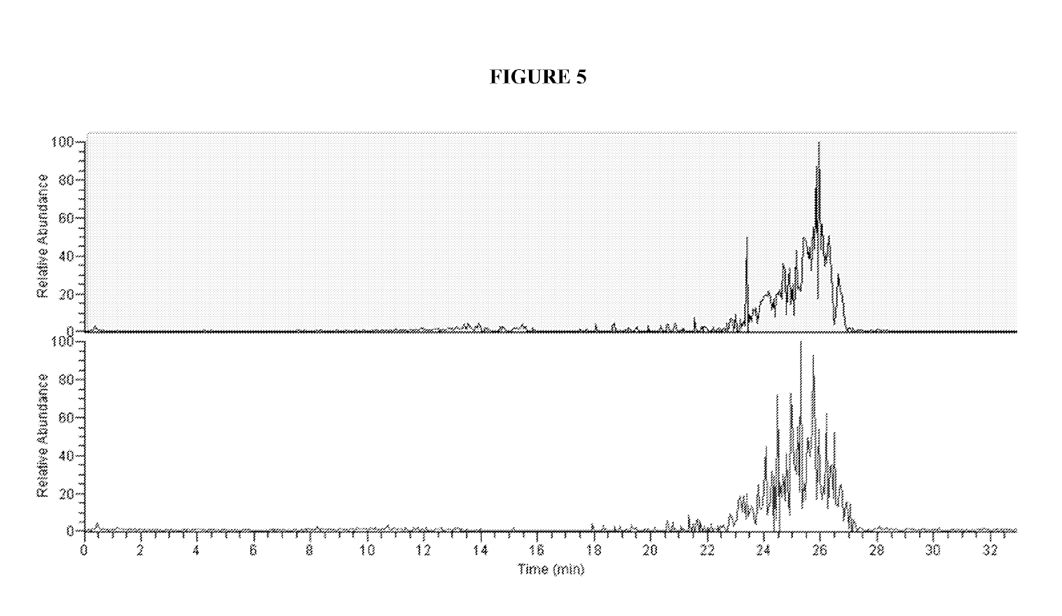

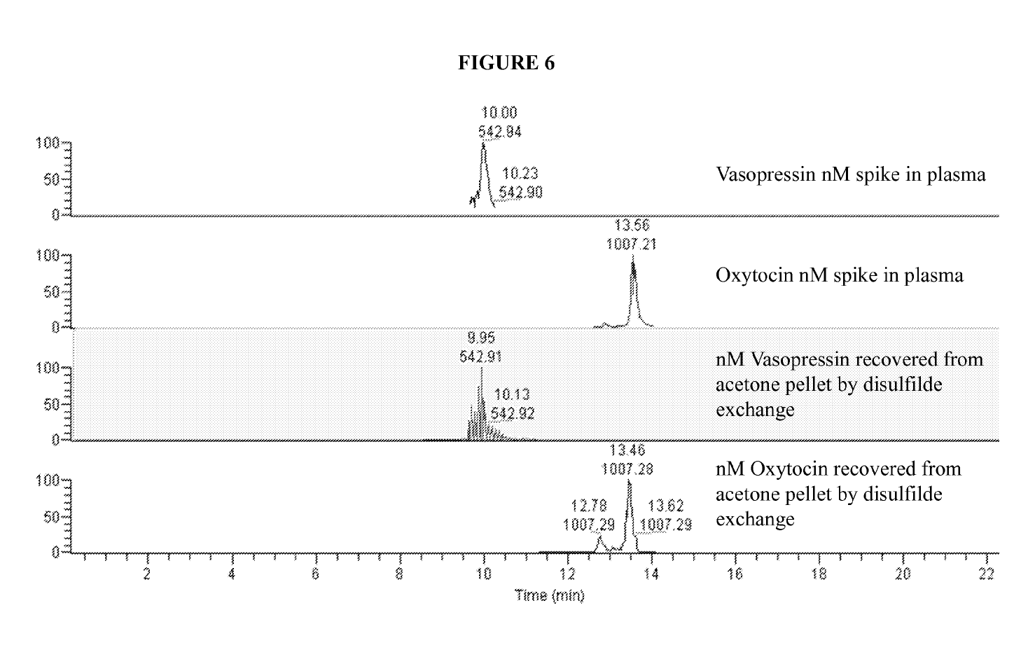

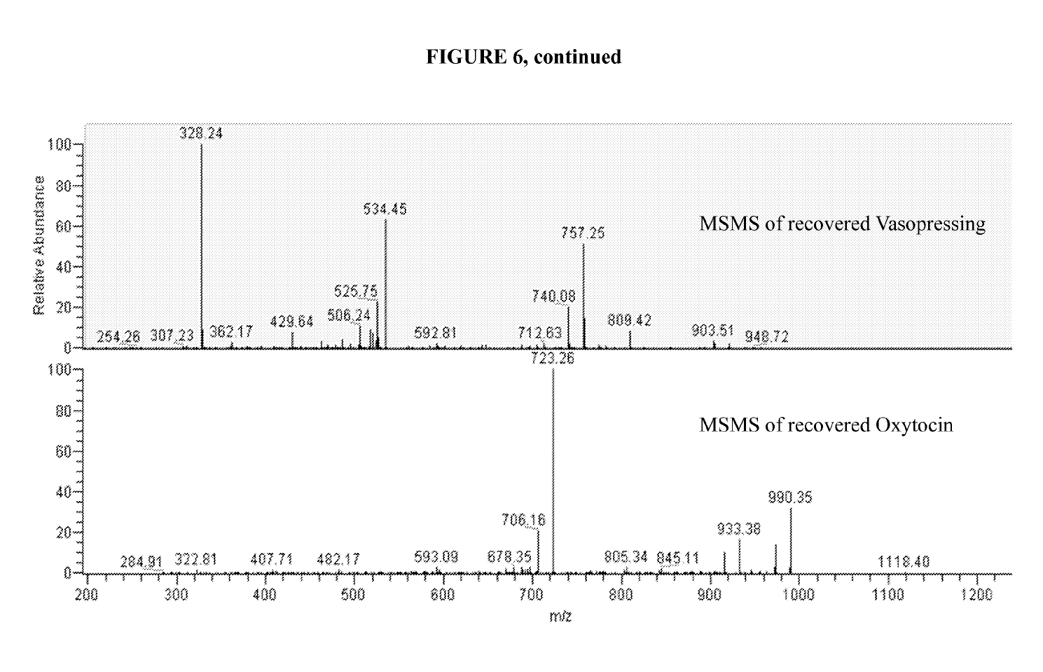

To further validate the reversible disulfide exchange sequestration of oxytocin and vasopressin exogenous hormones were added at mid-nanomolar levels to plasma and incubated the mixtures at room temperature for 20 minutes, followed by acetone extraction (FIG. 6). The acetone extracted pellets were washed, dissolved with guanidinium chloride and reducing agents at room temperature. Upon dissolution, the resuspensions were diluted and reprecipitated. The supernatents were simplified by solid phase extraction and analyzed by LC-MS/MS. Mid-nanomolar amounts of both hormones were recovered from the material that first pelleted with plasma proteins but was released from them under denaturing and reducing conditions.

Endogenous Oxytocin and Vasopressin Maybe Isolated from Plasma at nM Concentrations if Plasma Redox State is Perturbed:

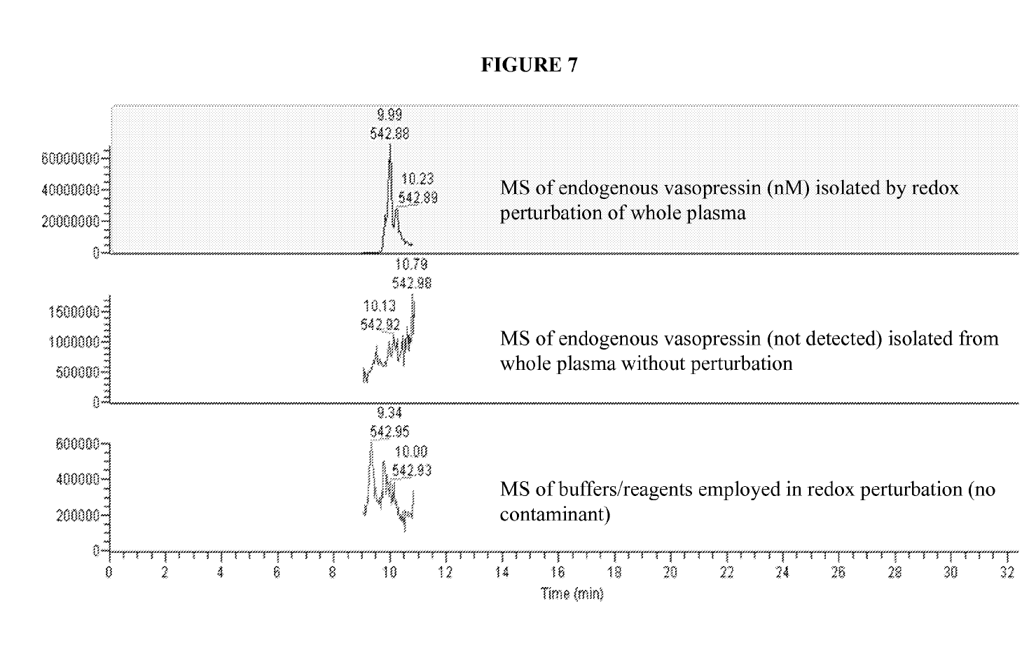

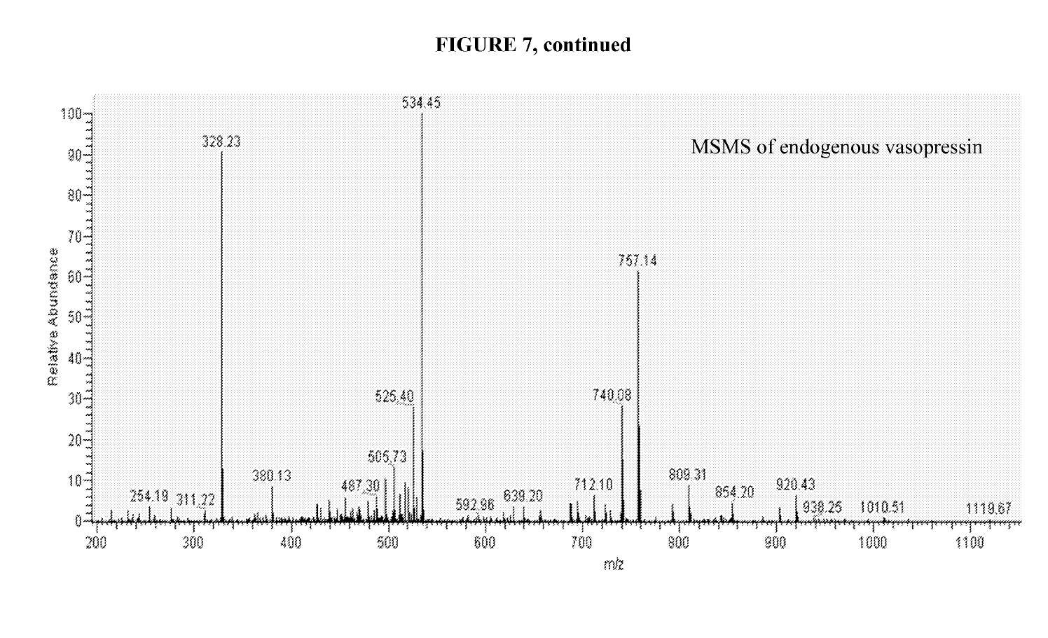

Whole plasma is modified to perturb the redox state (FIG. 7, top MS) or not (FIG. 6, middle MS) and processed to recover endogenous vasopressin. LC-MS/MS analysis reveals nM levels (ng/mL) of vasopressin in the redox perturbed sample and undetectable (low pg/mL) levels of vasopressin from plasma not perturbed. The bottom MS panel of FIG. 6 verifies the absence of vasopressin in the perturbation reagents.

Nanomolar levels (ng/mL) of vasopressin and oxytocin (results not shown) are higher than obtained from normal individuals with immunoreactivity, which was expected. Not all disulfide exchange adducts will be equally immunoreactive and protein bound forms may exhibit dramatically reduced immunoreactivity. An ensemble of reversibly interchanging pools of oxytocin and vasopressin in various redox and protein conjugated states will defy any attempt to quantify and enumerate the specific entities. Only the total quantity, when all interactions are disrupted and neutralized, is measurable. The total level obtained is more in line with the levels used in medical interventions and those required for response in biological activity assays.

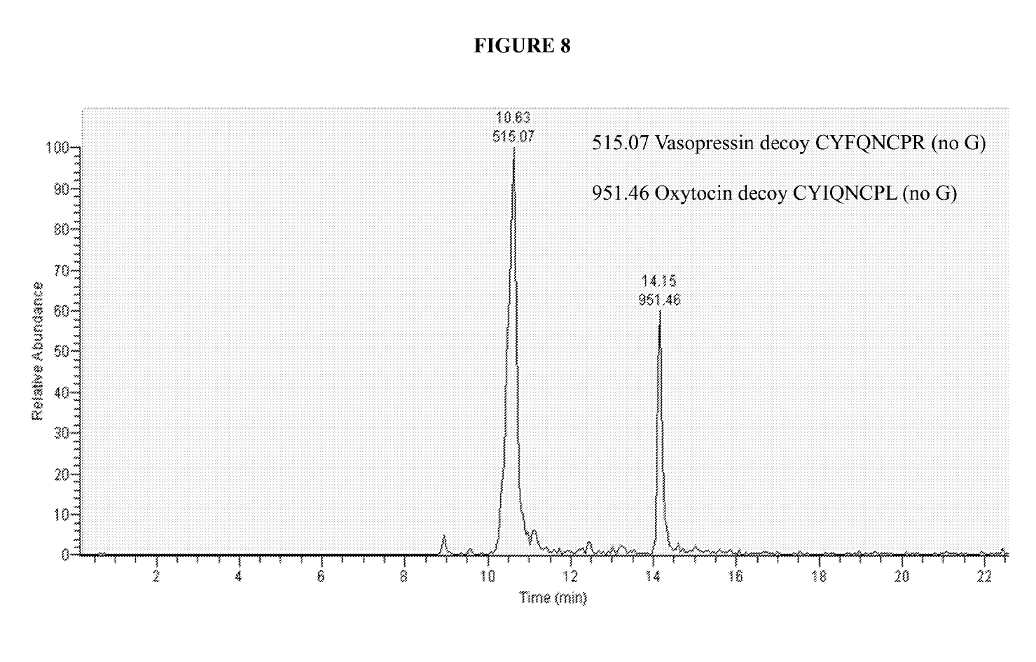

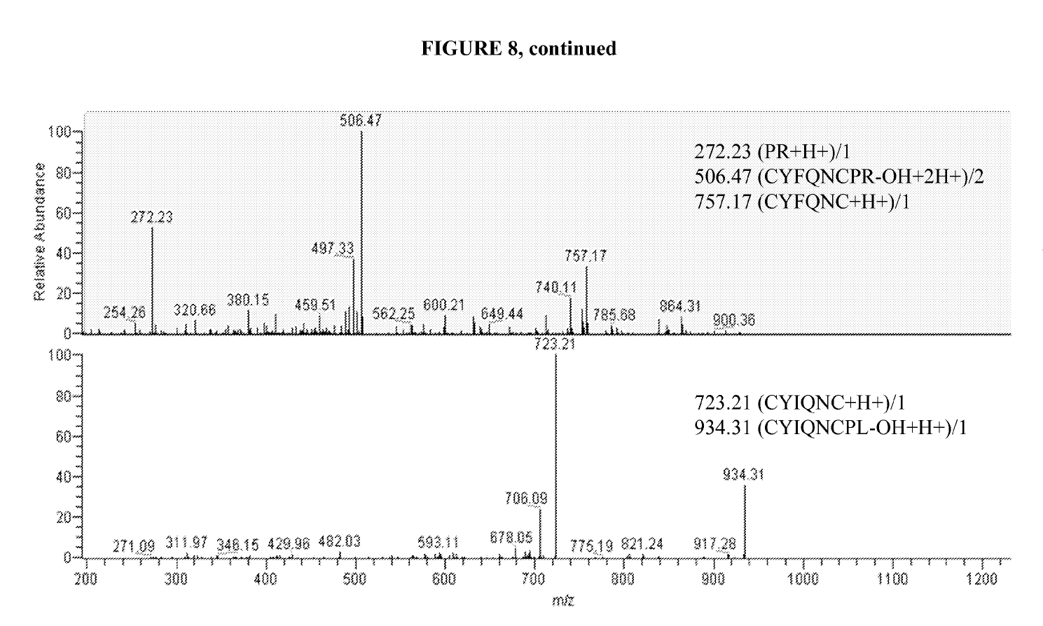

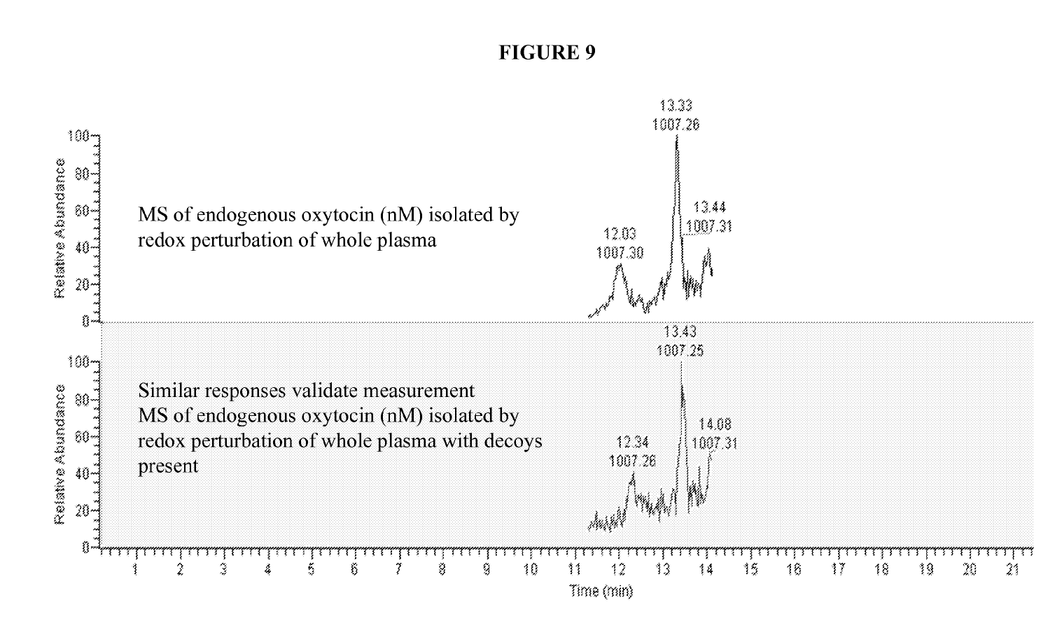

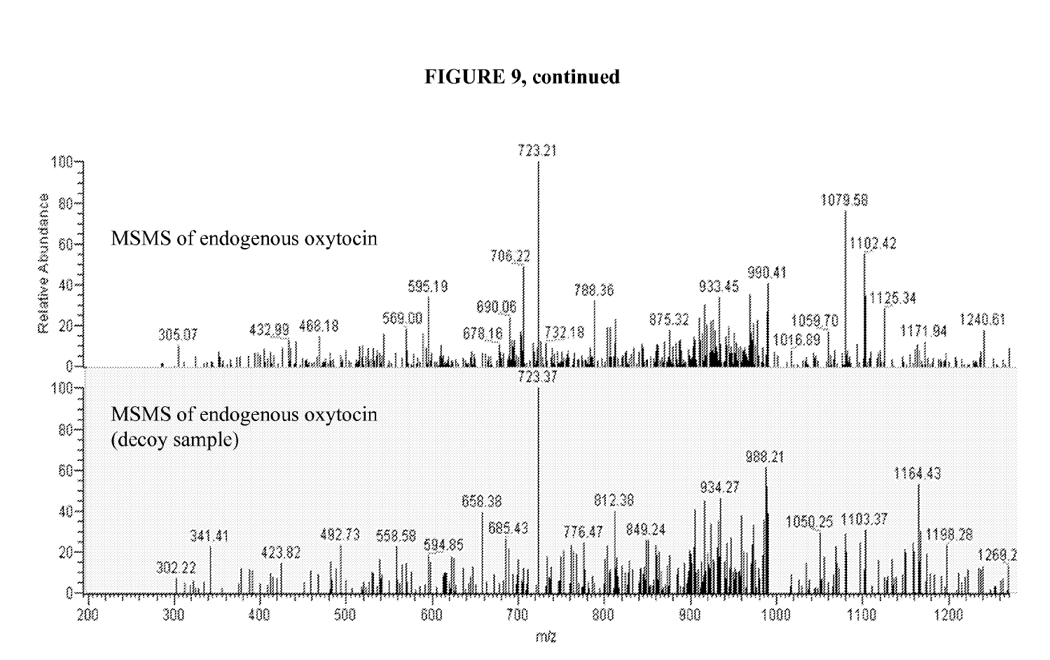

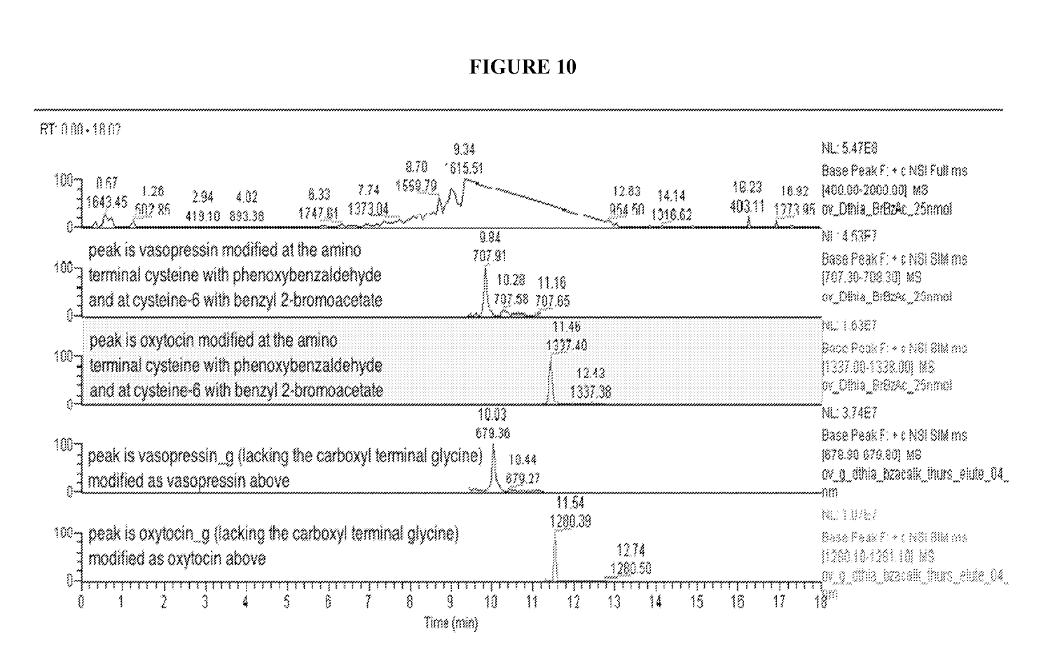

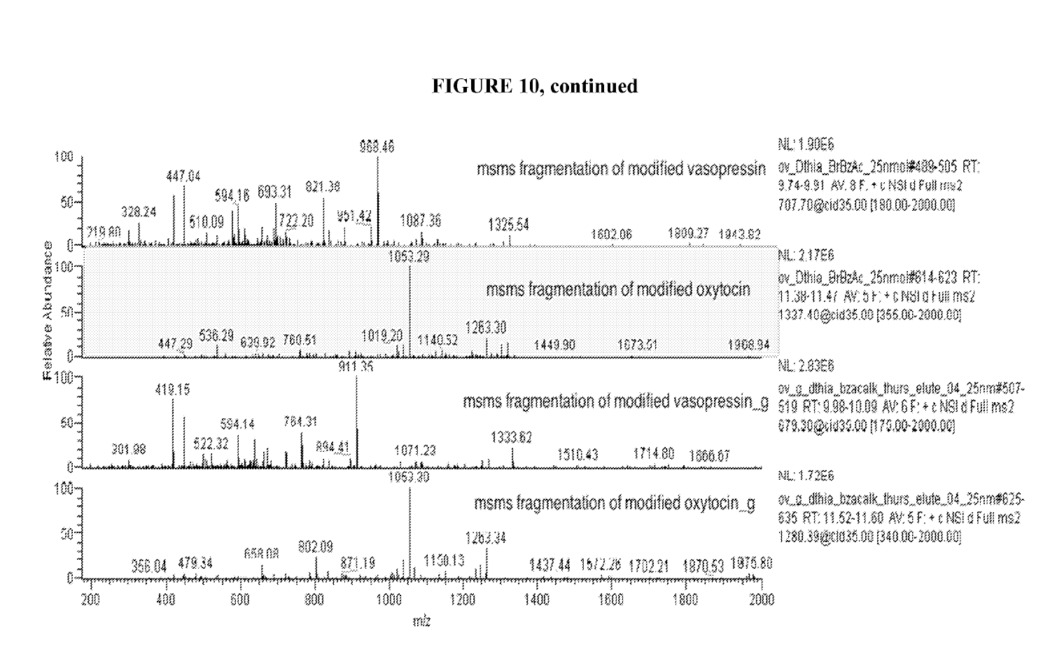

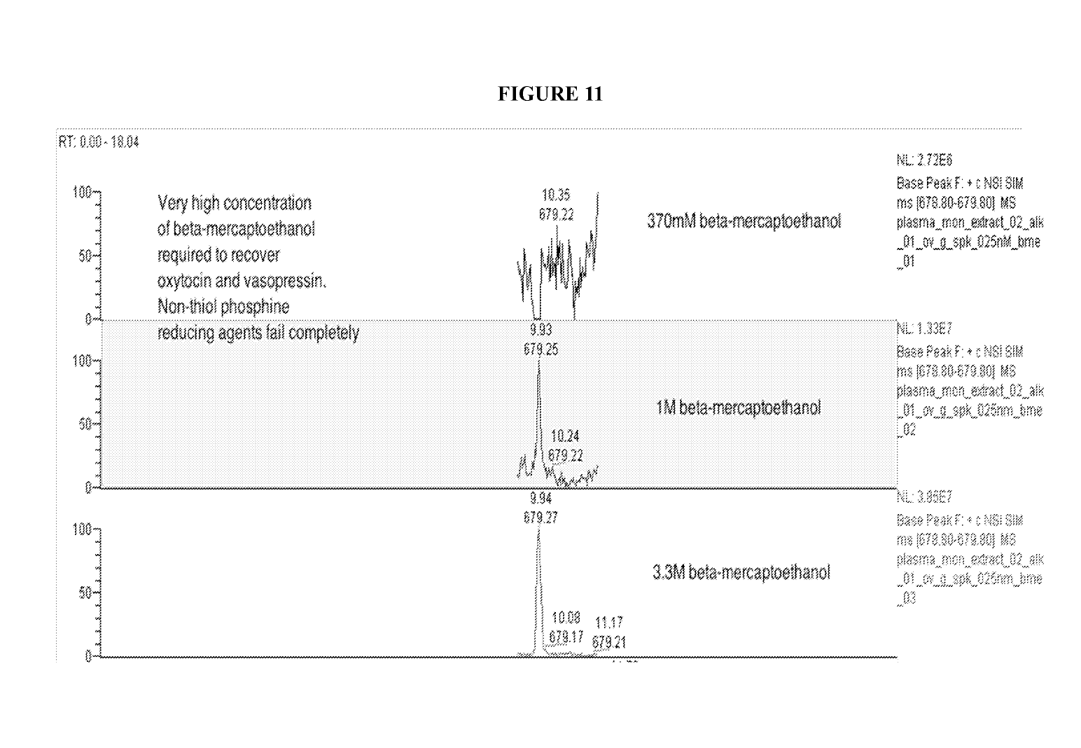

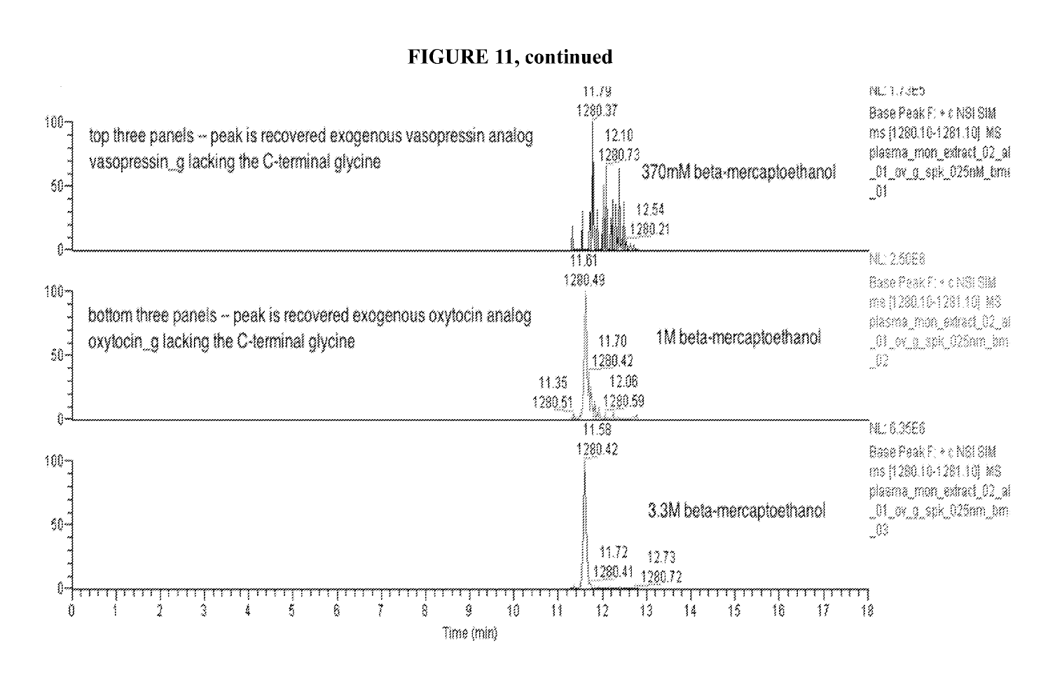

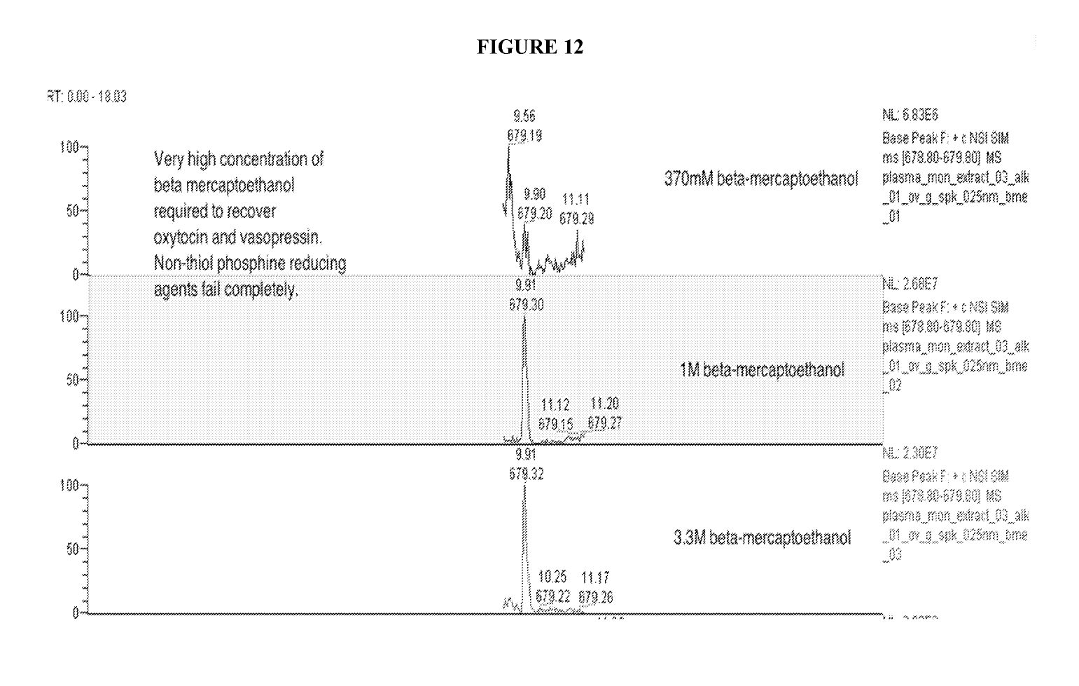

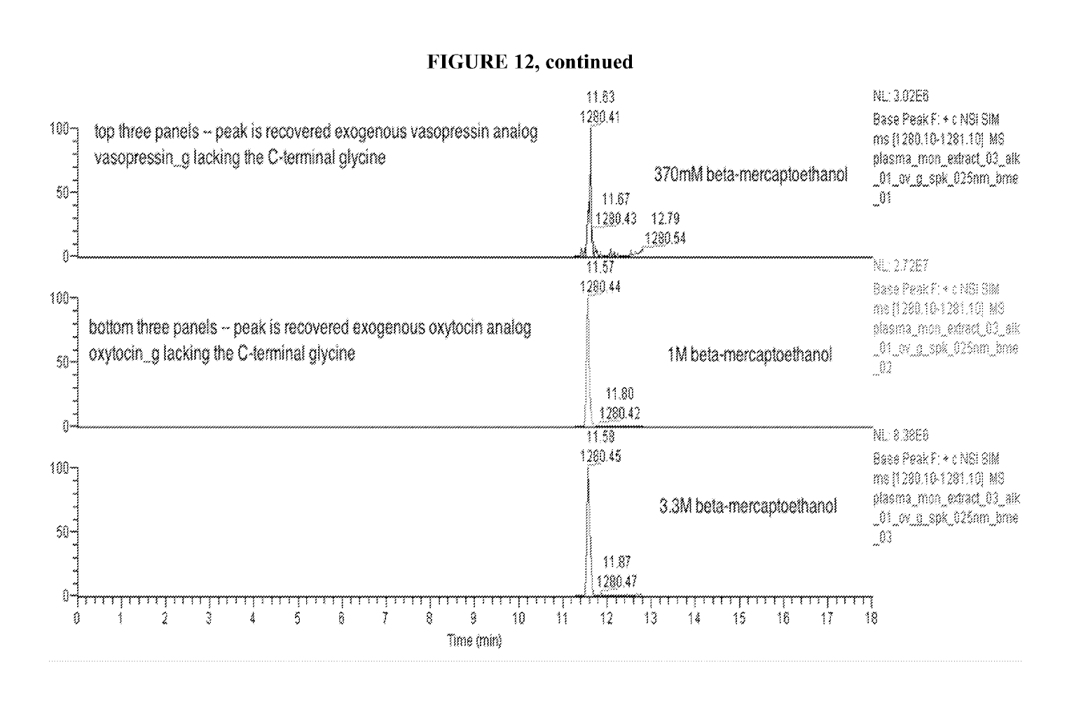

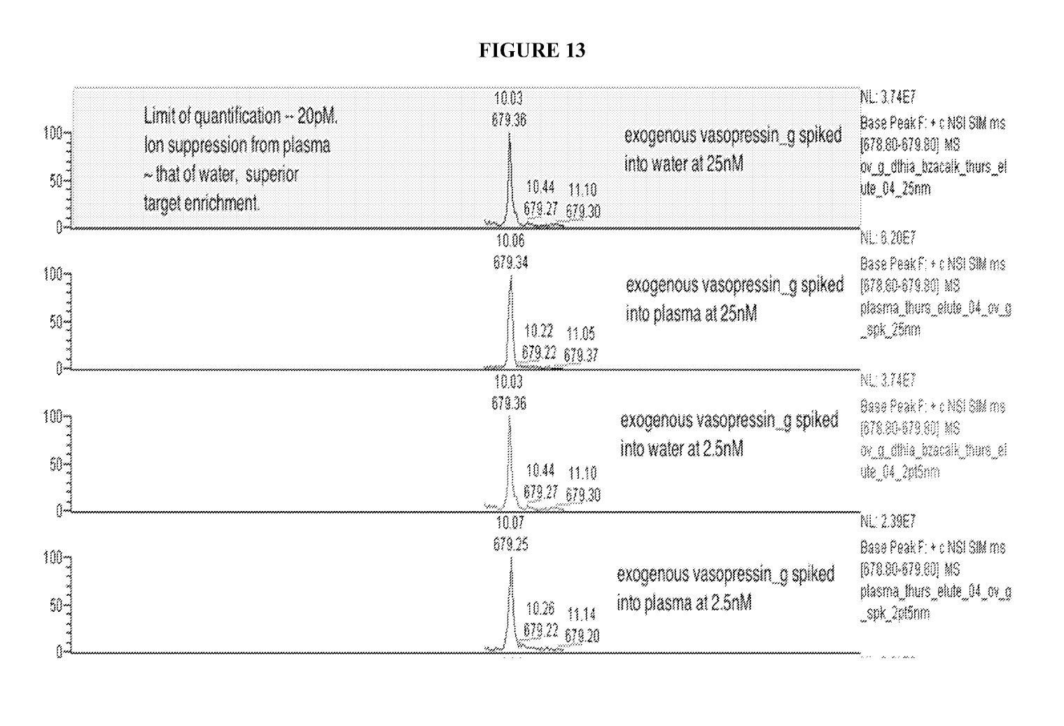

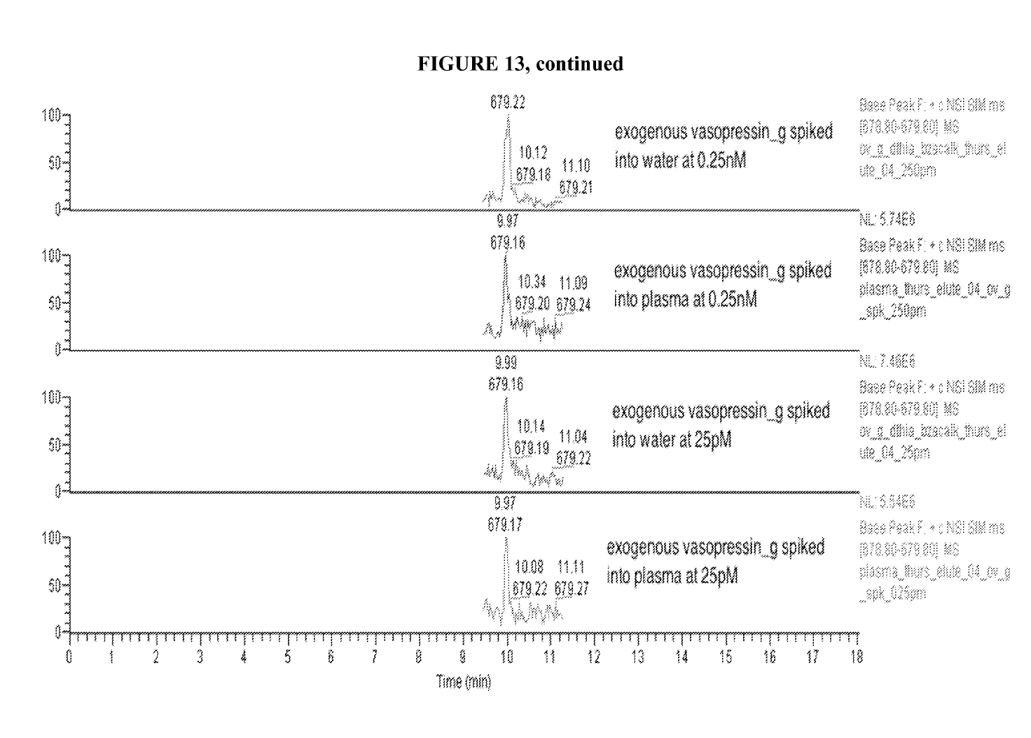

Perturbing the redox state correctly is challenging. To ensure complete recovery of oxytocin and vasopressin, the samples are treated with vast excess of analog peptides without C-terminal glycines. Decoy peptides (FIG. 8, in vast excess) that are capable of participating equally with target compounds in both disulfide exchange and protein binding are employed. The decoys elute the targets from the plasma if needed and validate the quality of the disruption achieved in their absence. When a protocol is employed without the decoy (FIG. 9, top) and in the presence of decoy (FIG. 9, bottom) the disruption is optimal and the measurement is valid.

Reversible disulfide exchange of oxytocin and vasopressin in plasma enables hormone sequestration (circulating in biologically inactive forms) and local control of hormone activity through redox state perturbation. This system allows neurohypophyseal hormones to respond instantly and locally to inflammation, wounds and changes in vascular endothelia.

### Example 4

### Isolating Oxytocin and Vasopressin from Apheresis Platelets in Plasma, Platelet Rich Plasma, Standard Plasma, Serum, Milk and Saliva

The example illustrates isolating oxytocin and vasopressin from apheresis platelets in plasma, platelet rich plasma, standard plasma, serum and saliva. The same procedure may be applied to isolating oxytocin and vasopressin from saliva, milk, sweat and urine.

A 2 mL polypropylene eppendorf tube was charged with 400 μL of apheresis platelets in plasma (or same volume of other sample) and 400 μL of 7M guandidinium hydrochloride and 30 μL beta-mercaptoethanol were added to give:

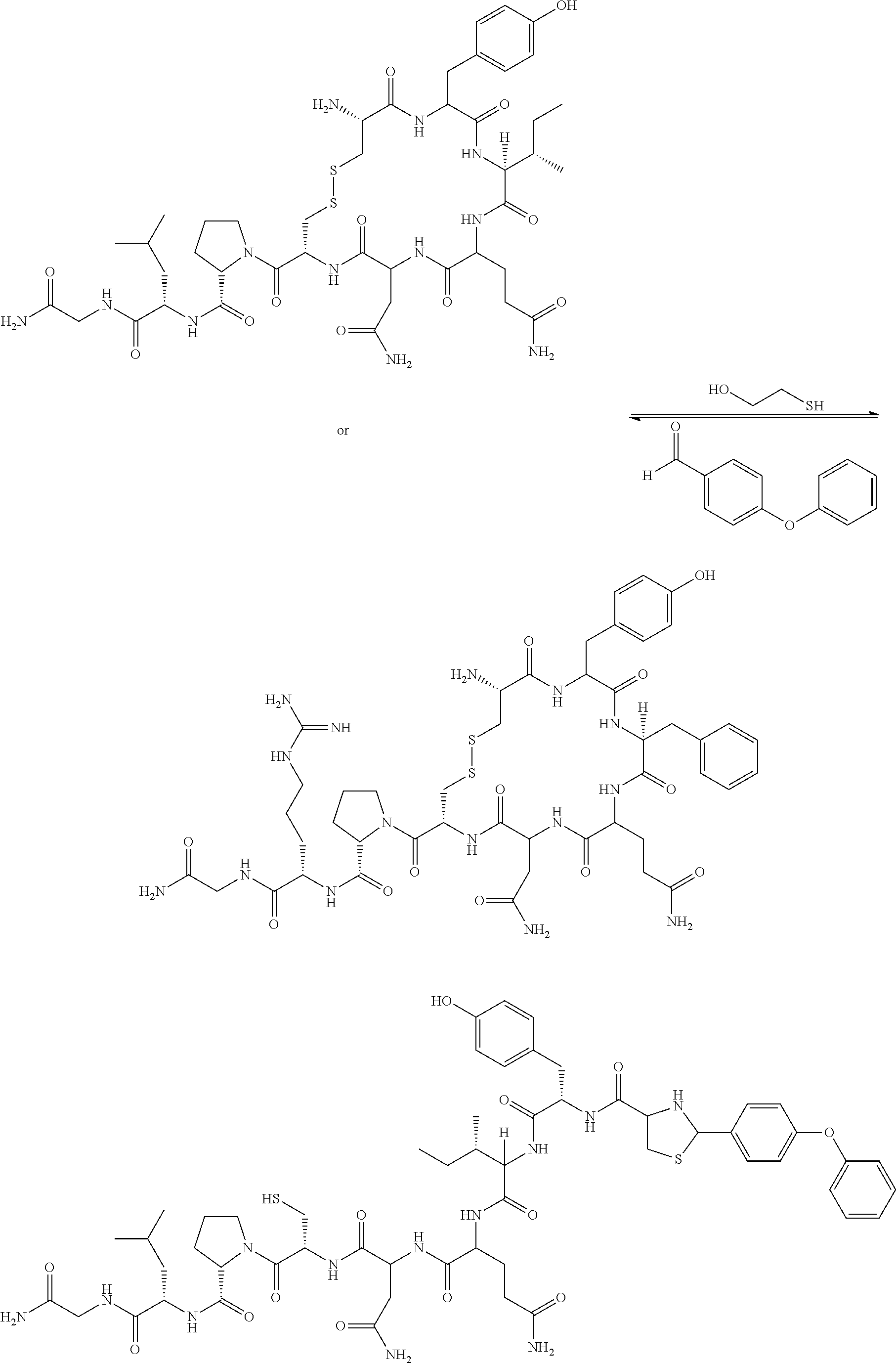

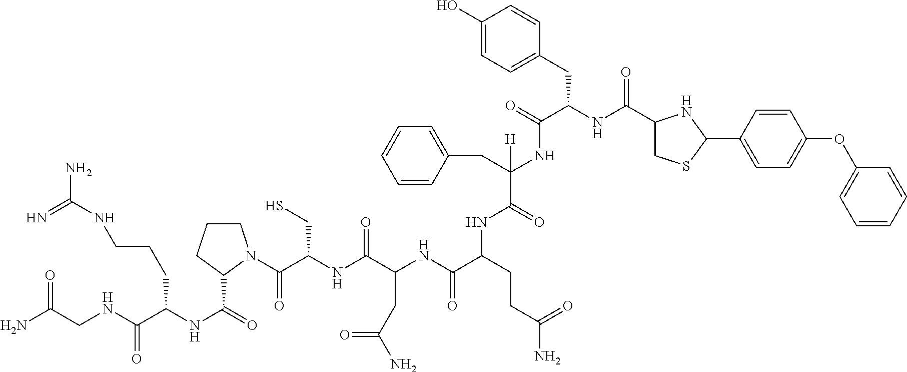

The mixture was then extracted with 800 μL ethyl acetate three times, discarding the organic layers. The mixture was further extracted with 700 μL acetonitrile+100 μL ethyl acetate three times discarding the organic layers. The aqueous mixture was transferred to a 2 mL glass vial, and added 100 μL dimethylformamide, 10 μL beta-mercaptoethanol, 10 μL 1M NaₓH_{y}PO₄ pH 7.4, 5 μL 200 mM triscarboxyethylphosphine (TCEP), 8 μL 1M 4-phenoxybenzaldehyde and vortexed at 1000 rpm overnight. Then, 40 μL 400 mM cysteine in 1M NaₓH_{y}PO₄ pH 7.4 was added and allowed to vortex for 1 hour. The mixture was transferred to a 2 mL polypropylene eppendorf tube, and thiazolidine reaction mixtures were extracted with 700 μL acetonitrile. The organic layer was concentrated it under nitrogen gas.

The wells on a 40 mg tC18 resin 96 well plate were wetted with 700 μL dioxane, 700 μL acetonitrile and 700 μL 1 mM NaxHyPO4 pH 7.4. 2 μL of 100 mM Benzyl 2-bromoacetate (acetonitrile) in 200 μL 1 mM NaxHyPO4 pH 7.4 was loaded onto each well.

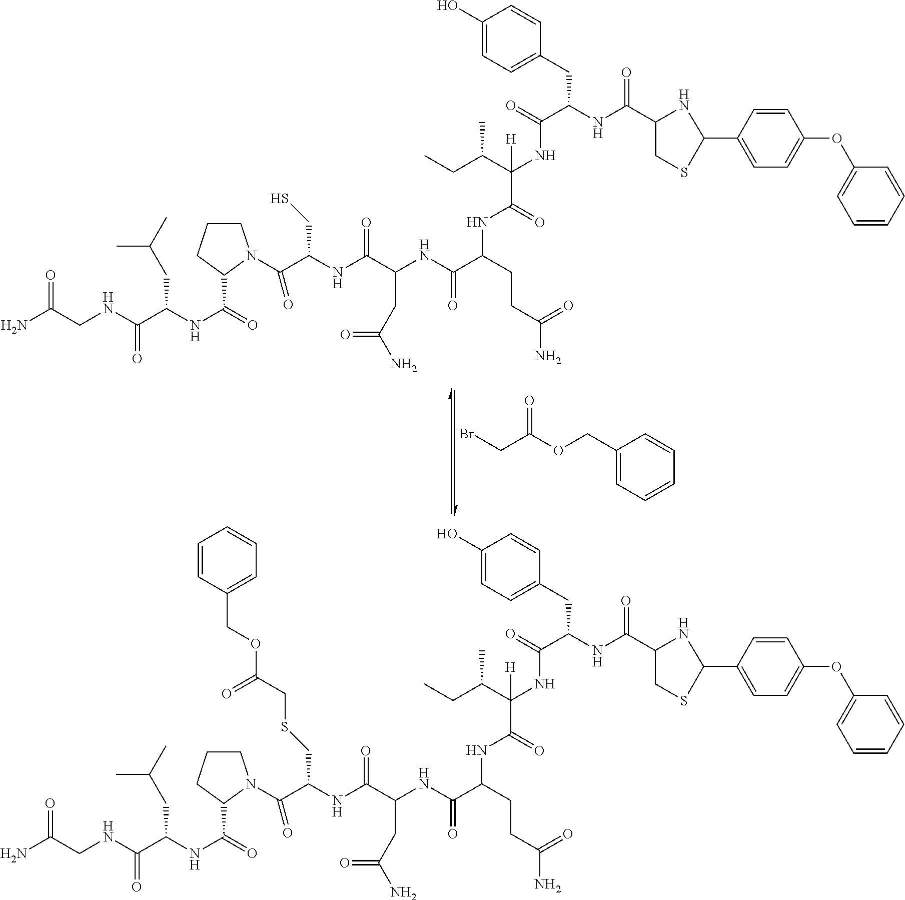

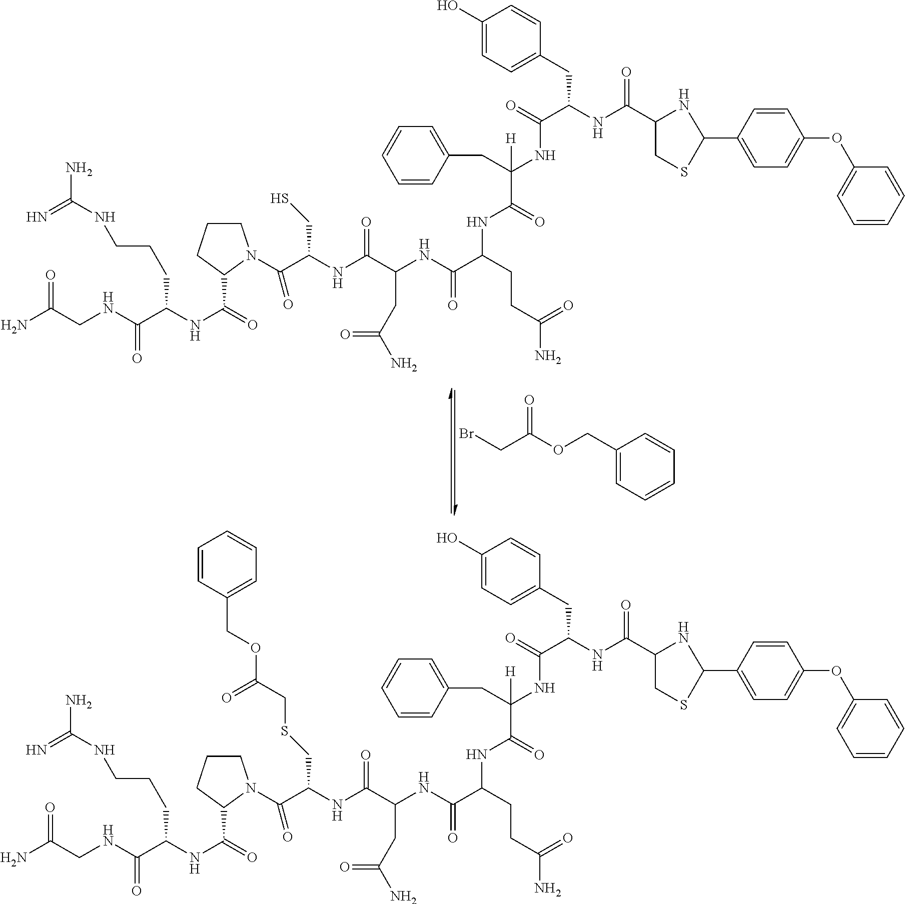

500 μL 10 mM NaₓH_{y}PO₄ pH 7.4 with 10% acetonitrile and TCEP 1 mM was added to each well, mixed in sample into this volume. The sample was allowed to load under gravity flow. Then, 500 μL 10 mM NaₓH_{y}PO₄ pH 7.4 with 10% acetonitrile and TCEP 1 mM and 2 μL 100 mM Benzyl 2-bromoacetate (acetonitrile) was added to each well; and the sample was allowed to load under gravity flow. Then, 500 μL 10 mM NaₓH_{y}PO₄ pH 7.4 with 10% acetonitrile and TCEP 1 mM and 2 μL 400 mM cysteine in 1M NaₓH_{y}PO₄ pH 7.4 was added to each well; and the sample was allowed to load under gravity flow.

Then, 400 μL 10 mM NaxHyPO4 pH 7.4 with 10% acetonitrile, was added, spun through filter for 3 minutes at 100 rpm at 15° C. on an Allegra 6 kR centrifuge. Flow through and remaining solvent on top of resin was removed and discarded. Then, 400 μL 10 mM NaₓH_{y}PO₄ pH 7.4 with 30% acetonitrile, was added and spun through filter for 6 minutes at 100 rpm at 15° C. on an Allegra 6 kR centrifuge. The flow through was removed and discarded, repeated until flow through volume ˜300 μL, then removed and discarded remaining solvent on top of resin. Then, 400 μL 10 mM NaₓH_{y}PO₄ pH 7.4 with 40% acetonitrile, was added and spun through filter for 4 minutes at 100 rpm at 15° C. on an Allegra 6 kR centrifuge. The flow through was removed and discarded, repeated until flow through volume ˜300 μL, then removed and discarded remaining solvent on top of resin. Then, 400 μL 10 mM NaₓH_{y}PO₄ pH 7.4 with 50% acetonitrile was added, spun through filter for 4 times 1 minute at 100 rpm at 15° C. on an Allegra 6 kR centrifuge. The flow through was removed and discarded, repeated until flow through volume ˜300 μL, then removed and discarded remaining solvent on top of resin. Targets are in second and third fractions, which were then pooled and concentrated under nitrogen gas, and analyzed by LC-MS/MS.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A method for processing a biological sample, comprising:
contacting the sample with one or more of reducing agents, denaturants, detergents, modification reagents, and/or agents capable of catalyzing oxidation;
precipitating the high-molecular weight substituents of the sample with organic solvent;
evaporation of the organic solvent; and
subjecting the aqueous remainder of the supernatent to solid phase extraction.

2. The method of claim 1, further comprising eluting a peptide from solid phase extraction resin.

3. A method for detecting oxytocin and vasopressin in a sample, comprising: processing the sample according to claim 2; and detecting oxytocin and vasopressin in the sample.

4. The method of claim 1, further comprising contacting the sample with a basic compound.

5. The method of claim 1, further comprising contacting the sample with an acidic compound.

6. The method of claim 1, where the denaturant is guanidinium hydrochloride.

7. The method of claim 1, where the detergent is triton x-100.

8. The method of claim 1, where the detergent is sodium dodecyl sulfate.

9. The method of claim 1, wherein the sample is a biological fluid.

10. The method of claim 9, wherein the biological fluid is blood, plasma, serum, saliva, urine, breast milk or cow's milk.

11. The method of claim 4, wherein the basic compound is sodium carbonate.

12. The method of claim 4, wherein the basic compound raises the pH of the sample to at least 10.

13. The method of claim 5, wherein the acidic compound is formic acid.

14. A method for determining the presence or an amount of a polypeptide in a biological sample, the method comprising:
a. adding to the sample a decoy polypeptide
b. contacting the sample with a reducing agent and an agent capable of catalyzing oxidation;
c. isolating the polypeptide and decoy from the sample, and
d. detecting the polypeptide in the eluent.

15. The method of claim 14, wherein the reducing agent is glutathione.

16. The method of claim 14, wherein the polypeptide is oxytocin or vasopressin.

17. The method of any one of claim 14, wherein the polypeptide lacks the C-terminal glycine.

18. The method of any one of claim 14, wherein the isolating comprise solid phase extraction.

19. The method of any one of claim 14, wherein the detecting comprises mass spectroscopy.

20. The method of any one of claim 14, wherein the detection comprises LC-MS.

21. A method for determining the presence or an amount of a polypeptide in a biological sample, the method comprising:
a. contacting the sample with a reducing agent and an agent capable of catalyzing oxidation to obtain a reduced polypeptide;
b. contacting the reduced polypeptide with an alkylating agent to obtain an alkylated polypeptide;
c. isolating the alkylated polypeptide from the sample, and
d. detecting the polypeptide in the eluent.

22. The method of claim 21, wherein the reducing agent is glutathione or β-mercaptoethanol.

23. The method of claim 21, wherein the reducing agent is β-mercaptoethanol.

24. The method of any one of claim 21, wherein the alkylating agent is an electron withdrawn aldehydes.

25. The method of claim 24, wherein the aldehyde is 4-phenoxy benzaldehyde.

26. The method of any one of claim 21, the polypeptide is oxytocin or vasopressin.

27. The method of any one of claim 21, wherein the polypeptide lacks the C-terminal glycine.

28. The method of any one of claim 21, wherein the isolating comprise solid phase extraction.

29. The method of any one of claim 21, wherein the detecting comprises mass spectroscopy (e.g., LC-MS)


SEQUENCE LISTING (7 sequences)

SEQ ID NO: 1 - protein, 9 aa, Artificial Sequence
  vasopressin
CYFQNCPRG

SEQ ID NO: 2 - protein, 9 aa, Artificial Sequence
  oxytocin
CYIQNCPLG

SEQ ID NO: 3 - protein, 12 aa, Artificial Sequence
  oxytocin dodecapeptide
CYIQNCPLGGKR

SEQ ID NO: 4 - protein, 6 aa, Artificial Sequence
  MS-MS fragment
CYFQNC

SEQ ID NO: 5 - protein, 6 aa, Artificial Sequence
  MS-MS fragment
CYIQNC

SEQ ID NO: 6 - protein, 8 aa, Artificial Sequence
  synthesized oxytocin analog
CYIQNCPL

SEQ ID NO: 7 - protein, 8 aa, Artificial Sequence
  the synthesized vasopressin analog
CYFQNCPR